(19) United States
(12) Patent Application Publication
(10) Pub. No.: US 2025/0171517 A1
(43) Pub. Date: May 29, 2025

(54) METHODS OF PRODUCING HUMAN ANALOG INSULINS AND DERIVATIVES THEREOF IN A MAMMALIAN CELL

(71) Applicant: Ice Bear Therapeutics SPC, Seattle, WA (US)

(72) Inventors: Christian PFLEGER, Seattle, WA (US); Andreas PFLEGER, Singen (DE)

(21) Appl. No.: 18/837,132

(22) PCT Filed: Feb. 9, 2023
(86) PCT No.: PCT/US2023/062257

§ 371 Date: Aug. 8, 2024

Related U.S. Application Data
(60) Provisional application No. 63/308,913, filed on Feb. 10, 2022.

(51) Int. Cl. C07K 14/62; A61K 38/28; C12N 9/48; C12N 9/64; C12N 15/85; C12P 21/02

(52) U.S. Cl. CPC C07K 14/62; A61K 38/28; C12N 9/485; C12N 9/6454; C12N 15/85; C12P 21/02; C12N 2310/20; C12Y 304/1701; C12Y 304/21093; C12Y 304/21094

(57) ABSTRACT

There is a need for alternative methods for recombinant expression of functional insulin and/or insulin analogs having its native conformation. The present disclosure provide methods for producing functional, recombinant protein having its native conformation in mammalian host cell cultures wherein the recombinant protein is an insulin analog and/or a derivative thereof.

### CROSS REFERENCE

The present application claims priority to and benefit from U.S. Provisional Application No. 63/308,913 filed Feb. 10, 2022, the entire contents of which are herein incorporated by reference.

### DESCRIPTION OF THE XML FILE SUBMITTED ELECTRONICALLY

The contents of the electronic sequence listing (IBTS_001_01WO_SeqList_ST26.xml; Size: 32,369 bytes; and Date of Creation: Feb. 6, 2023) are herein incorporated by reference in their entirety.

### TECHNICAL FIELD

The present disclosure generally relates to methods for producing functional, recombinant protein in its native conformation. The invention is particularly useful for the production of proteins that benefit post-translational modifications such as trimming, proper folding and glycosylation.

### DESCRIPTION OF THE RELATED ART

Insulin is an important polypeptide hormone, produced by pancreatic beta cells of the islets of Langerhans, and is required to maintain blood glucose homeostasis in mammals, including humans, and other vertebrates. In a healthy individual, an increase in blood glucose level stimulates the β-cells of the pancreas to secrete insulin. The insulin polypeptide then binds to specific receptors in muscle, liver, and adipose tissue leading to an increase in glucose uptake by these targeted tissues, an increase in metabolism, and a decrease in hepatic glucose production. The cumulative effects of these responses serve to keep blood glucose concentrations at a constant level.

About 177 million people around the world suffer from Diabetes mellitus. These include about 17 million type I diabetics for whom replacement of the lacking endocrine insulin secretion is the only possible therapy at present, without which complication like ketoacidosis, coma, and death within weeks can occur. Type I diabetics treated with suboptimal doses of insulin develop secondary diseases such as ketoacidosis, blindness, heart attack, and kidney failure. Despite tremendous progress in transplantation and cell therapy, there is still no cure in sight for type 1 diabetes. Type II diabetes contrasts with type I diabetes in that there is relative, not absolute deficiency of insulin, but in a large number of cases, especially in the advanced stage, treatment with insulin therapy is inevitable, and is the most favorable type of therapy. In addition, according to estimates, the number of individuals diagnosed with diabetes will double to approximately 300 million, in the next 25 years (Kjeldsen, T. et al., 2001, Biotechnol. Gen. Eng. Rev. 18:89-121). However, access to insulin therapy comes at a high cost as the average cost of insulin in the USA has increased over the years. Consequently, the ability to cost effectively manufacture human insulin in quantities to satisfy the anticipated growing world demand for insulin is highly desirable.

Since the development of recombinant DNA technology, numerous methods have been described to produce insulin, precursors, and analogs thereof in genetically modified host cells. Many production systems for recombinant proteins have become available, ranging from bacteria, yeasts, and insect cells to plant cells and mammalian cells. However, the existing production systems and methods for recombinant insulin production suffer from various disadvantages. Prokaryotic production systems routinely employed for the recombinant production of insulin analogs, such as Escherichia coli (Frank et al., 1981, in Peptides: Proceedings of the 7^{th} American Peptide Chemistry Symposium (Rich & Gross, eds.), Pierce Chemical Co., Rockford. Ill pp 729-739; Chan et al., 1981, Proc Natl. Acad. Sci. USA 78: 5401-5404), Saccharomyces cerevisiae (Thim et al., 1986, Proc. Natl. Acad. Sci. USA 83: 6766-6770), are unable to provide correct folding of the expressed polypeptide or establish the disulfide bond connecting the A- and B-chains in the mature insulin. Eukaryotic systems provide an improvement, but still suffer from a number of drawbacks. The hypermannosylation in, for instance, yeast strains affects the ability of yeasts to properly express glycoproteins. Moreover, hypermannosylation often even leads to immune reactions when a therapeutic protein thus prepared is administered to a patient. Furthermore, yeast secretion signals are different from mammalian signals, leading to a more problematic transport of mammalian proteins, including human polypeptides, to the extracellular, which in turn results in problems with continuous production and/or isolation.

Wang et al. (Biotechnol. Bioeng., 2001, 73:74-79) have shown that yeast, such as Pichia pastoris, are suitable for insulin production. However, one of the major disadvantages of this system is the post-translational modification of resulting proteins, which later exist as impurities in the final product that is difficult to purify. Additionally, enzymatic steps utilized in the existing production systems are time consuming, costly and introduce additional impurities that subsequently have to be removed in further downstream process steps like expensive chromatography steps and the like.

Mammalian cells are widely used for the production of proteins because of their ability to perform extensive post-translational modifications. However, recombinant production of glycoproteins or proteins comprising at least two (different) subunits in mammalian cells continues to be a challenge.

Given the complex methods, high cost, and low yields of existing production systems and methods of recombinant production and purification of biologically active insulin and or insulin analogs, there is a need for alternative methods for recombinant expression of functional insulin and/or insulin analogs having its native conformation. Presently disclosed embodiments address these needs and provide other related advantages.

### SUMMARY

Provided herein are methods and compositions for producing functional, recombinant protein in its native conformation in host cell cultures. The method of the present disclosure comprises transforming the host cells with at least one expression vector to co-express a protein precursor (i.e. pro-protein or pro-peptide) and at least one agent (e.g. protease) for processing the protein precursor to a functional protein by post-translational modification (e.g. enzymatic cleavage). Non-limiting examples of the protein that can be produced by the methods of the present disclosure include insulin, amylin, gastrin, ghrelin, glucagon, somatostatin, α-MSH, ACTH, β-endorphin, or other peptide hormones. In some aspects, the host cells are mammalian cells selected from a group consisting of HEK293 cells, CHO cells, COS, and HeLa cells. In other aspects, the host cells are selected from a group consisting of algae and yeast.

According to the present disclosure, the recombinant protein comprises mature insulin or insulin analogs. In particular, the new and improved methods of the present disclosure relate to a process for preparing at least one insulin analog and/or insulin derivative that offers the advantages of a higher yield and higher purity of desired product, while reducing the number of steps required for obtaining the at least one insulin analog and/or insulin derivative. The methods disclosed herein pertain to a highly efficient process for the production of recombinant human insulin analogs and/or derivatives thereof that reduces and/or eliminates the presence of undesirable cleavage by-products, and that further presents the advantages of eliminating several time consuming and expensive, purification steps.

In some aspects, the present disclosure provides improved methods for the production of insulin analogs or derivatives thereof. In certain aspects, the present disclosure provides methods for the production of insulin analogs or derivatives thereof in a mammalian host cell. Advantageously, the methods and compositions according to the present disclosure are able to utilize the more evolved abilities of mammalian cell host machinery to read, fold, and cleave the expressed recombinant insulin analog, in vivo. Further, the methods and compositions of the present disclosure utilize agents that enable in vivo processing of recombinant insulin analog, and are exogenously introduced into the mammalian cell host, thereby enabling the production of mature insulin analog, without the additional steps of digestion, processing, and purification, to optimize and simplify the process for producing functional insulin and/or insulin analogs. The optimized methods and compositions of the present disclosure translate into saved costs in materials, equipment, and time, as well as improved yield through lowering protein loss in comparison to current/existing production methods

Accordingly, the present disclosure provides a method for producing a functional recombinant protein in its native conformation comprising an insulin analog and/or derivative thereof in mammalian host cells. In certain aspect, the method comprises providing competent mammalian host cells. According to the present disclosure, the method comprises transforming the competent mammalian host cells with an expression vector suitable for expression in mammalian host cells. The expression vector comprises a heterologous nucleic acid sequence encoding a precursor peptide and/or a modified human pro-insulin peptide for at least one human insulin analog or derivatives thereof. In certain aspects, the method comprises culturing a colony of the transformed mammalian host cells in a suitable growth media under conditions suitable for the expression and secretion of the precursor peptide and/or a modified human proinsulin peptide for the at least one human insulin analog or derivatives thereof; and harvesting or recovering the secreted insulin analog and/or derivative thereof. In certain aspects, the recovery of the secreted peptide comprises recovering the mature processed peptide from the growth media. In any of the presently disclosed aspects, the secreted insulin analog and/or derivative thereof is a functional insulin analog and/or derivative thereof in its native conformation and is capable of binding to an insulin receptor peptide.

The present disclosure provides an expression vector comprising in the 5′ to 3′ direction of transcription as operably linked components: (i) a nucleic acid sequence capable of controlling expression in a mammalian host cell; (ii) a nucleic acid sequence encoding a signal peptide for efficient secretion of insulin analog or derivative thereof; (iii) optionally, a nucleic acid sequence encoding a N-terminal fusion partner; (iv) a nucleic acid sequence encoding a modified pro-insulin polypeptide; and (v) a nucleic acid sequence encoding one or more agents for processing the modified pro-insulin to functional insulin analog or derivatives thereof. The agent for processing the modified pro-insulin to functional insulin analog or derivatives thereof is selected from a group consisting of Prohormone Convertase 1/3 (PC1/3), PC2 and carboxypeptidase E (CPE). In some aspects, the agents for processing the modified pro-insulin to functional insulin lispro or derivatives thereof comprise PC1/3, CPE, and, optionally, PC2. In another aspect, the agents for processing the modified pro-insulin to functional insulin glargine or derivatives thereof comprise PC1/3 and, optionally, PC2.

The present disclosure provides a first and a second expression vectors. In some aspects, the first expression vector comprises in the 5′ to 3′ direction of transcription as operably linked components: (i) a nucleic acid sequence capable of controlling expression in mammalian cells; (ii) a nucleic acid sequence encoding a signal peptide for efficient secretion of insulin analog or derivative thereof, (iii) optionally, a nucleic acid sequence encoding a N-terminal fusion partner; and (iv) a nucleic acid sequence encoding a modified pro-insulin polypeptide. In another aspect, the second expression vector comprises in the 5′ to 3′ direction of transcription as operably linked components: (i) a nucleic acid sequence capable of controlling expression in mammalian cells; and (ii) a nucleic acid sequence encoding one or more agents for processing the modified pro-insulin to functional insulin analog or derivatives thereof. The agent for processing the modified pro-insulin to functional insulin analog or derivatives thereof is selected from the group consisting of PC1/3, PC2 and CPE. In some aspects, the agents for processing the modified pro-insulin and/or pro-insulin analog to functional insulin lispro or derivatives thereof comprise PC1/3, CPE, and optional PC2. In another aspect, the agents for processing the modified pro-insulin to functional insulin glargine or derivatives thereof comprise PC1/3, and, optionally, PC2.

The present disclosure provides a heterologous nucleic acid sequence encodes a precursor peptide and/or modified proinsulin peptide for the at least one human insulin analog, wherein the heterologous nucleic acid sequence is prepared by altering the pro-insulin gene through replacement of codon(s) at the appropriate site in the native human pro-insulin gene. In certain aspects, the replacement of codon(s) encodes a desired amino acid residue substitute(s). Non-limiting representative examples of desired amino acid substitutions are B28Lys, B29Pro human insulin, B28Asp human insulin, A21Gly human insulin, B31Arg, B32Arg human insulin, B31Leu, B32Ala human insulin; B31Leu, B32Ala, desB30 human insulin, B31Phe, B32Leu human insulin and B31Phe, B32Leu, desB30 human insulin.). According to the present disclosure, the heterologous nucleic acid is prepared by synthesizing the whole DNA-sequence encoding the at least one human insulin analog.

In certain aspects, the heterologous nucleic acid encoding the modified proinsulin peptide and/or the precursor peptide of the at least one human insulin analog or derivatives thereof encodes the B-chain of human insulin or an analog thereof, the A-chain of human insulin or an analog thereof, and a C-peptide linking the B-chain and the A-chain together.

According to the present disclosure, the heterologous nucleic acid sequence encoding the precursor peptide for the at least one human insulin analog or derivatives thereof further comprises nucleic acid sequences encoding one or more agents for processing the modified proinsulin to functional insulin analog or derivatives thereof. In certain aspects, the one or more agents for processing the modified proinsulin to functional insulin analog or derivatives thereof comprise PC 1/3, PC2, CPE and a blocking agent effective in blocking endogenous CPE activity. In certain aspects, the mammalian host cell is modified to express the one or more agents for processing the modified proinsulin to functional insulin analog or derivatives thereof.

The present disclosure provides a method comprises transforming the competent mammalian host cells with a first expression vector comprising the heterologous nucleic acid sequence encoding the precursor peptide and/or the modified human proinsulin peptide for the at least one human insulin analog or derivatives thereof, and with a second expression vector comprising a heterologous nucleic acid sequence encoding one or more agents for processing the modified proinsulin to functional insulin. In certain aspects, the one or more agents for processing the modified pro-insulin to functional insulin comprise endoproteases, carboxypeptidase E, and a blocking agent effective in blocking endogenous carboxypeptidase E activity. In an aspect, the endoproteases comprise the PC1/3 and PC2. In certain aspects, the mammalian host cell is modified to express the one or more agents for processing the modified proinsulin to functional insulin analog or derivatives thereof.

In certain aspects, the heterologous nucleic acid sequence encodes a peptide that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 1, SEQ ID: No. 2; SEQ ID: No. 3, and SEQ ID: No. 4.

In certain aspects the heterologous nucleic acid sequence encodes a peptide that has an amino acid sequence as set forth in SEQ ID: No. 1, SEQ ID: No. 2; SEQ ID: No. 3, and SEQ ID: No. 4.

According to the present disclosure, the heterologous nucleic acid further encodes one or more agents for processing the modified pro-insulin to functional insulin. In some aspects, the one or more agents has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 5, SEQ ID: No. 6; SEQ ID: No. 7, and SEQ ID: No. 8. In certain aspects, the one or more agents has an amino acid sequence as set forth in SEQ ID: No. 5, SEQ ID: No. 6; SEQ ID: No. 7, and SEQ ID: No. 8.

The heterologous nucleic acid sequence encoding the at least one insulin analog or derivative thereof is inserted into a suitable expression vector which when transferred to a suitable host organism expresses the desired product. The expressed product is secreted from the host cell and recovered from the culture broth.

In certain aspects, the present disclosure provides a polynucleotide encoding a peptide comprising a modified human proinsulin or a human pro-insulin analog or derivatives thereof, and one or more agents for processing the modified proinsulin to functional insulin. In certain aspects, the heterologous nucleic acid sequence encoding a peptide comprising a modified human proinsulin or a human pro-insulin analog or derivatives thereof has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 1, SEQ ID: No. 2; SEQ ID: No. 3, and SEQ ID: No. 4. In some aspects, the modified human proinsulin or a human pro-insulin analog or derivatives thereof encoded by the peptide has an amino acid sequence as set forth in SEQ ID NO:1, SEQ ID NO:2; SEQ ID NO:3, and SEQ ID NO:4. In certain aspects, the polynucleotide further comprises a heterologous nucleic acid sequence encoding one or more agents for processing the modified proinsulin to functional insulin. In certain aspects, the polynucleotide further comprises a heterologous nucleic acid sequence encoding a blocking agent effective in blocking endogenous carboxypeptidase E activity. The one or more agents for processing the modified proinsulin to functional insulin comprise one or more endoproteases and carboxypeptidase E. The one or more endoproteases are selected from PC1/3, PC2. In some aspects, the one or more agents has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 5, SEQ ID: No. 6; SEQ ID: No. 7, and SEQ ID: No. 8. In certain aspects, the one or more agents has an amino acid sequence as set forth in SEQ ID: No. 5, SEQ ID: No. 6; SEQ ID: No. 7, and SEQ ID: No. 8.

The present disclosure also pertains to an expression vector and a modified mammalian host cell comprising the polynucleotide encoding a peptide comprising a modified human proinsulin or a human pro-insulin analog or derivatives thereof and at least one agent for processing the modified proinsulin to functional insulin. In some aspects, the mammalian host cell comprises a genetic modification to facilitate formation of stable disulfide bonds within the cytoplasm for processing the modified pro-insulin to functional insulin. In certain aspects, the present disclosure also provides a human cell comprising an expression vector encoding a peptide comprising a modified human proinsulin or a human insulin analog or derivatives thereof. In certain aspects, the present disclosure also provides a human cell comprising an expression vector encoding a peptide comprising a modified human proinsulin or a human pro-insulin analog or derivatives thereof and the at least one agent for processing the modified proinsulin to functional insulin.

In some aspects, the present disclosure provides methods for manufacturing insulin analog and/or a derivative thereof comprising producing mammalian host cells that express and secret insulin comprising transforming the mammalian host cells with an expression vector co-expressing a modified pro-insulin polypeptide and one or more agents for processing the modified pro-insulin to functional insulin analog or derivatives thereof. In another aspects, the present disclosure provides methods for manufacturing insulin analog and/or a derivative thereof comprising producing competent mammalian host cells that make and secret insulin comprising transforming the host cells with a first expression vector expressing a pro-insulin polypeptide and a second expression vector expressing one or more agents for processing the pro-insulin and/or pro-insulin analog to functional insulin analog or derivatives thereof.

In some aspects, the present disclosure provides a method for manufacturing rapid-acting insulin analog and/or a derivative thereof comprises transforming mammalian host cells with a recombinant expression vector comprising (i) a nucleic acid sequence capable of controlling expression in a mammalian host cell; (ii) a nucleic acid sequence encoding a signal peptide for efficient secretion of insulin analog or derivative thereof; (iii) optionally, a nucleic acid sequence encoding a N-terminal fusion partner; (iv) a nucleic acid sequence encoding a modified pro-insulin polypeptide; (v) a nucleic acid sequence encoding PC1/3; (vi) a nucleic acid sequence encoding CPE; and (vii) optionally, a nucleic acid sequence encoding PC2. In some aspects, the rapid-acting insulin analog is insulin lispro.

In some aspects, the present disclosure provides a method for manufacturing long-acting insulin analog and/or a derivative thereof comprises transforming mammalian host cells with a recombinant expression vector comprising (i) a nucleic acid sequence capable of controlling expression in a mammalian host cell; (ii) a nucleic acid sequence encoding a signal peptide for efficient secretion of insulin analog or derivative thereof; (iii) optionally, a nucleic acid sequence encoding a N-terminal fusion partner; (iv) a nucleic acid sequence encoding a modified pro-insulin polypeptide; (v) a nucleic acid sequence encoding PC1/3; and (vi) optionally, a nucleic acid sequence encoding PC2. In some aspects, the long-acting insulin analog is insulin glargine.

In some aspects, the methods for manufacturing long-acting insulin analog and/or a derivative thereof further comprises the optional step of blocking endogenous carboxypeptidase E (CPE) activity. In some aspects, the step of blocking endogenous carboxypeptidase E activity comprises introducing a deletion or mutation into endogenous CPE that results in reduced or eliminated expression or activity. In some aspects, the host cell may be a CRISPR-modified host cell having reduced or eliminated CPE activity. Such host cells may be produced, for example, by reducing or eliminating expression of CPE using CRISPR or by permitting expression of a CPE that is catalytically-inactive. For example, the serine at position 202 of the sequence set forth in SEQ ID NO: 9 may be mutated, for example, to Proline. In some aspects, the reduction or elimination of CPE activity may be conditional or inducible (e.g. in the form of Cre dependent constructs). In some aspects, the step of blocking endogenous carboxypeptidase E activity comprises co-expressing a mutated CPE with at least one mutation at a position selected from a group consisting of 72H, 75E, 147R, 192H, 202S, 243Y, and 296E of the sequence set forth in SEQ ID NO: 9 in the mammalian host cells. In some aspects, a nucleic acid sequence encoding the mutated CPE is included in the expression vector co-expressing a modified pro-insulin polypeptide. In another aspect, a nucleic acid sequence encoding the mutated CPE is included in a separate expression vector. In some aspects, the present disclosure provides a host cell expressing a catalytically-inactive CPE, wherein the CPE binds substrate but does not cleave a peptide bond, wherein the catalytically-inactive CPE has at least one mutation at a position selected from a group consisting of 72H, 75E, 147R, 192H, 202S, 243Y, and 296E of the sequence set forth in SEQ ID NO: 9. In some aspects, the step of blocking endogenous CPE activity comprises adding a blocking agent selected from a group consisting of dopamine quinine, dopamine, norepinephrine, epinephrine, potato carboxypeptidase inhibitor (PCI), 9-mer peptide-designated CPI-2KR, and a peptide encoding a decoy arginine sequence. The decoy arginine peptide of the present disclosure comprises a polymer of at 3 amino acids in length and having an amino acid sequence with at least 70% amino acid sequence identity to the C-terminal extension of the B chain of a long-acting insulin, for example, the sequence set forth in SEQ ID NO: 15. The decoy arginine peptide is configured to mimic the C-terminal extension of the B-chain of a long-acting insulin to bind endogenous CPE so that it block the endogenous CPE from removing the C-terminal extension of the B-chain.

In some aspects, the present disclosure pertains to a method for obtaining a purified biologically active heterologous recombinant protein expressed in a mammalian expression system. In certain aspects, the method comprises culturing the host cells transformed by at least one vector comprising a heterologous nucleic acid sequence encoding a peptide that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: NO. 1, SEQ ID: NO. 2; SEQ ID: NO. 3 or SEQ ID: NO. 4, in a suitable growth culture media and conditions suitable for the expression of the recombinant protein. In certain aspects, the method comprises culturing the host cells transformed by at least one vector comprising the nucleotide sequence encoding a peptide that has an amino acid sequence as set forth in SEQ ID NO: 1, SEQ ID NO: 2, SEQ ID NO: 3 or SEQ ID: No. 4. In certain aspects, the vector comprising the heterologous nucleic acid encoding the peptide further encodes one or more agents for processing the heterologous protein to a mature functional protein. In some aspects, the one or more agents has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: NO. 5, SEQ ID: NO. 6; SEQ ID: NO. 7, or SEQ ID: NO. 8. In certain aspects, the one or more agents has an amino acid sequence as set forth in SEQ ID: NO. 5, SEQ ID: NO. 6; SEQ ID: NO. 7, or SEQ ID: NO. 8.

In certain aspect, the method comprises recovering or harvesting the expressed recombinant protein, wherein the step of recovering the recombinant protein comprises separating the protein from the culture media to produce a recovered recombinant protein preparation. In some aspects, the method further comprises purifying the recovered recombinant protein preparation to remove at least one related impurity by contacting the recovered protein preparation with a chromatographic matrix. Purification is carried out by applying a polar organic buffer solvent in the aqueous phase containing the organic acid buffer and precipitating the eluted protein. In any of the presently disclosed aspects, the mature functional recombinant protein is human insulin analog and/or derivative thereof. In any of the presently disclosed aspects, the recombinant human insulin comprises an A chain having the amino acid sequence as set forth in SEQ ID NO: 10, and a B chain having the amino acid sequence as set forth in SEQ ID NO: 11. In another aspect, the recombinant human insulin comprises an A chain having the amino acid sequence set forth in SEQ ID NO: 12, and a B chain having the amino acid sequence set forth in SEQ ID NO: 13. Still in another aspect, the recombinant human insulin comprises an A chain having the amino acid sequence set forth in SEQ ID NO: 14, and a B chain having the amino acid sequence set forth in SEQ ID NO: 14. In certain aspects, the recombinant human insulin comprises an A chain having the amino acid sequence at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: NO. 10, and a B chain having the amino acid sequence at least about 85% (i.e., at least about 85%, 86%, 87%, 89%, 90%, 91%, 92%, 93%, 94%, 95%, 96%, 97%, 98%, 99%, or more) identity to the amino acid sequence as set forth in SEQ ID NO: 11. In certain aspects, the recombinant human insulin comprises an A chain having the amino acid sequence at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 12, and a B chain having the amino acid sequence at least about 85% (i.e., at least about 85%, 86%, 87%, 89%, 90%, 91%, 92%, 93%, 94%, 95%, 96%, 97%, 98%, 99%, or more) identity to the amino acid sequence as set forth in SEQ ID NO: 13. In certain aspects, the recombinant human insulin comprises an A chain having the amino acid sequence at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 14, and a B chain having the amino acid sequence at least about 85% (i.e., at least about 85%, 86%, 87%, 89%, 90%, 91%, 92%, 93%, 94%, 95%, 96%, 97%, 98%, 99%, or more) identity to the amino acid sequence as set forth in SEQ ID NO: 15.

According to the current disclosure, the yield of the purified insulin analog and/or derivative thereof is 75%-100%. According to another aspect of the present disclosure, the yield of the purified insulin analog and/or derivative thereof is 75%-80%, 80%-85%, 85%-90%, 90%-95%, or 95%-100%.

The present disclosure provides an integrated and continuous process for manufacturing a recombinant human insulin analog and/or derivative thereof, the process comprising: (i) culturing recombinant human insulin analog secreting mammalian cells in a perfusion bioreactor comprising a liquid culture medium under conditions that allow the cells to secrete the recombinant the insulin analog and/or derivative thereof into the medium, and wherein substantially cell-free volumes of the medium are continuously or periodically removed from the perfusion bioreactor and fed into a first periodic counter current chromatography system (PCCS1); (ii) capturing the recombinant insulin analog and/or derivative thereof from the medium using the PCCS1, wherein an eluate of the PCCS1 comprising the recombinant insulin analog and/or derivative thereof is continuously fed into a second periodic counter current chromatography system (PCCS2); and (iii) purifying the recombinant insulin analog and/or derivative thereof using the PCCS2, wherein the purifying is performed using a resin in the PCCS2 that is different in chemical structure compared to the resin in the PCCS2 used to perform the polishing, and an eluate from the PCCS2 is the insulin analog and/or derivative thereof; and wherein the process is integrated and runs continuously from the culturing step to the eluate from the PCCS2 that is the insulin analog and/or derivative thereof. In certain aspects, the recombinant human insulin analog-secreting mammalian cells are transformed by at least one vector comprising a heterologous nucleic acid sequence encoding a peptide that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: NO. 1, SEQ ID: NO. 2; SEQ ID: NO. 3 or SEQ ID: NO. 4. In certain aspects, the recombinant human insulin analog-secreting mammalian cells are transformed by at least one vector comprising a heterologous nucleic acid sequence encoding a peptide comprising the amino acid sequence as set forth in SEQ ID NO: 1, SEQ ID NO: 2, SEQ ID NO: 3, or SEQ ID: No. 4.

The human insulin analogs and derivatives thereof according to the present disclosure are useful in the treatment of states that are sensitive to insulin. Thus, the human insulin analogs and derivatives thereof may be used in the treatment of type 1 diabetes, type 2 diabetes and hyperglycemia for example as sometimes seen in seriously injured persons and persons who have undergone major surgery.

In a further aspect, the present disclosure relates to pharmaceutical formulations comprising the human insulin analogs in combination with suitable pharmaceutically acceptable adjuvants and additives such as one or more agents suitable for stabilization, preservation or isotonicity.

Accordingly, in yet another aspect the present disclosure provides a method of treating diabetes in a subject in need thereof, said method comprising administering to said subject a therapeutically effective amount of the human insulin analog obtained by the methods disclosed herein.

Other features and advantages of the present disclosure will become readily apparent from the following detailed description. It should be understood, however, that the detailed description and the specific examples while indicating preferred aspects, are given by way of illustration only, since various changes and modifications within the spirit and scope of the disclosure will become readily apparent to those skilled in the art from this detailed description.

### BRIEF DESCRIPTION OF THE DRAWINGS

The embodiments disclosed herein will become more fully apparent from the following description and appended claims, taken in conjunction with the accompanying drawings.

FIG. 1 depicts the history of insulin production and further provides a schematic overview of the synthesis strategy of insulin analogs by the methods of the present disclosure. Details of the procedure are provided in Examples 1-3.

FIGS. 2A-2B shows the processing sites for the production of the mature insulin analog in a mammalian cell.

FIG. 3 describes insulin synthesis in pancreatic β-cells.

FIG. 4 shows small scale production (50 ml) of Lispro and Glargine harvested from 20× concentrated supernatant of HEK cells transformed with vectors comprising heterologous nucleic acid sequences encoding the respective insulin analogs and agents (Furin) required for processing of the recombinant protein to mature functional insulin analogs.

FIG. 5 shows the superiority of agents utilized by the human body (PC1/3, PC2, and Carboxypeptidase E) in comparison to Furin, to cleave the alpha, beta, and c-chain of recombinant insulin to produce mature insulin for the manufacture of Lispro insulin analog.

FIG. 6 the superiority of agents utilized by the human body (PC1/3, PC/2, and a mutant Carboxypeptidase E) in comparison to Furin, to cleave the alpha, beta, and c-chain of recombinant insulin to produce mature insulin for the manufacture of Glargine insulin analog.

FIGS. 7A-7D shows mass-spectrometric analysis of insulin analog Lispro (Scn-lispro) produced using: PC1 and CPE (non reduced)(7A); PC1 and CPE (reduced)(7B); PC1(non-reduced)(7C); and PC1(reduced)(7D).

FIGS. 8A-8D shows mass-spectrometric analysis of insulin analog Glargine produced by the methods of the present disclosure using: PC1 and mutant CPE (non reduced)(8A); PC1 and mutant CPE (reduced)(8B); PC1(non-reduced)(8C); and PC1(reduced)(8D).

### DETAILED DESCRIPTION

It will be readily understood that the embodiments, as generally described herein, are exemplary. The following description of various embodiments is not intended to limit the scope of the present disclosure, but is merely representative of various embodiments. Moreover, those skilled in the art may change the order of steps or actions of certain methods disclosed herein without departing from the scope of the present disclosure. In other words, unless a specific order of steps or actions is required for proper operation of the embodiment, the order or use of specific steps or actions may be modified.

Prior to setting forth this disclosure in more detail, it may be helpful to an understanding thereof to provide definitions of certain terms to be used herein. Additional definitions are set forth throughout this disclosure.

Unless defined otherwise, all technical and scientific terms used herein shall have the same meaning as is commonly understood by one skilled in the art to which the present disclosure belongs. Where permitted, all patents, applications, published applications, and other publications, including nucleic acid and polypeptide sequences from GenBank, SwissPro and other databases referred to in the disclosure are incorporated by reference in their entirety.

In the present disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non limiting.

“Optional” or “optionally” means that the subsequently described element, component, event, or circumstance may or may not occur, and that the description includes instances in which the element, component, event, or circumstance occurs and instances in which they do not.

As used herein, the term “prodrug” and/or “pro-insulin” and/or “precursor peptide”, is defined as any compound that undergoes chemical modification before exhibiting its pharmacological effects.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

Additionally, “amino acid” encompasses any molecule containing both amino and carboxyl functional groups, wherein the amino and carboxylate groups are attached to the same carbon (the alpha carbon). The alpha carbon optionally may have one or two further organic substituents. For the purposes of the present disclosure designation of an amino acid without specifying its stereochemistry is intended to encompass either the L or D form of the amino acid, or a racemic mixture. However, in the instance where an amino acid is designated by its three letter code and includes a superscript number, the D form of the amino acid is specified by inclusion of a lower case d before the three letter code and superscript number (e.g., dLys⁻¹), wherein the designation lacking the lower case d (e.g., Lys⁻¹) is intended to specify the native L form of the amino acid. In this nomenclature, the inclusion of the superscript number designates the position of the amino acid in the insulin analog sequence, wherein amino acids that are located within the insulin analog sequence are designated by positive superscript numbers numbered consecutively from the N-terminus. Additional amino acids linked to the insulin analog peptide either at the N-terminus or through a side chain are numbered starting with 0 and increasing in negative integer value as they are further removed from the insulin analog sequence. For example, the position of an amino acid within a dipeptide prodrug linked to the N-terminus of an insulin analog is designated aa⁻¹-aa⁰-insulin analog, wherein aa⁰ represents the carboxyl terminal amino acid of the dipeptide and aa⁻¹ designates the amino terminal amino acid of the dipeptide.

As used herein, “protein”, “polypeptide”, or “peptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein an “effective” amount or a “therapeutically effective amount” of an insulin analog refers to a nontoxic but sufficient amount of an insulin analog to provide the desired effect. For example, one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present disclosure may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

Unless specifically indicated otherwise, as used herein, a sequence identity of “at least about” an indicated percentage includes the indicated percentage±20% thereof, and every integer and non-integer percentage above the specific percentage. Accordingly, “at least about 85%” identity to the referenced sequence (e.g., any one of SEQ ID NOs.:1-4) includes about 85%, 86%, 87%, 89% 90%, 91%, 92%, 93%, 94%, 95%, 96%, 97%, 98%, 99%, or 100% identity to the referenced sequence, and also includes all non-integer percentages in between two integer percentages (e.g., 92.5%, 99.1%, etc.).

The terms “nucleic acid sequence encoding insulin” and “nucleic add sequence encoding an insulin analog” or “nucleic acid sequence encoding insulin polypeptide or a modified insulin peptide” or “pro-insulin”, which may be used interchangeably herein, refer to any and all nucleic add sequences encoding an insulin polypeptide, including but not limited to, the pro-insulin polypeptides listed in Table 2 (SEQ ID NO: 1, SEQ ID: NO.:2, SEQ ID: NO.:3, and SEQ ID: No. 4) as well as any mammalian insulin polypeptide and any nucleic add sequences that encode pro-insulin, prepro-insulin peptides and analogs thereof and/or derivatives thereof. The term “agents for processing” refer to any and all nucleic acid sequences encoding agents required for processing pro-insulin to mature functional insulin, including but not limited to, the polypeptides listed in Table 3 (SEQ ID NO: 5, SEQ ID: NO.:6, SEQ ID: NO.:7, and SEQ ID: No. 8).

In vivo the human insulin polypeptide is produced as a single 110 amino acid polypeptide chain precursor, pre-pro-insulin, which includes an N-terminally located 24 amino acid pre-sequence that is cleaved immediately upon completion of the chain's biosynthesis to yield pro-insulin (Steiner, D. F. 2000. J. Ped. Endocrinol. Metab. 13:229-239). Pro-insulin has the structure B-C-A, wherein a C-peptide chain connects the C-terminal amino acid of the B-chain with the N-terminal amino acid residue in the A-chain. During packaging of the hormone for secretion, the C-peptide is cleaved and removed by prohormone convertases, PC2 and PC1/PC3 (Steiner, D. F. 2000. J. Ped. Endocrinol. Metab. 13:229-239), yielding the mature or active human insulin, a 51 amino acid protein consisting of two polypeptide chains, A (21 amino acids in length) and B (30 amino acids in length), linked by two inter-chain disulphide bonds. The A- and B-chain are held together by two disulphide bridges between the A7 and B7 and the A20 and B19 Cys residues, respectively. In addition, the biologically active insulin molecule has an internal (intra-chain) disulphide bridge between the Cys residues in the position A6 and A11.

Nucleic acid sequences encoding an insulin polypeptide or peptide further include any and all nucleic add sequences which (i) encode polypeptides that are substantially identical to the insulin polypeptide sequences set forth herein; or (ii) hybridize to any nucleic acid sequences set forth herein under at least moderately stringent hybridization conditions or which would hybridize thereto under at least moderately stringent conditions but for the use of synonymous codons.

The term “pro-insulin” as used herein is a protein of the formula, wherein: A is the A chain of insulin or a functional derivative thereof, B is the B chain of insulin or a functional derivative thereof having an .epsilon.-amino group; and C is the connecting peptide of pro-insulin. Preferably, pro-insulin is the A chain of human insulin, the B chain of human insulin, and C is the natural connecting peptide. When pro-insulin is the natural sequence, pro-insulin possesses three free amino groups: Phenylalanine(1) (alpha.-amino group), Lysine(29) (.epsilon.-amino group) and Lysine(64) (epsilon.-amino group). As used herein “pro-insulin” refers to an insulin polypeptide, which includes the connecting peptide or “C-peptide” linking the B and A insulin polypeptide chains. In native human insulin, the C-peptide is the 31 amino acid residue polypeptide chain connecting residue B30 to residue A1. The term “prepro-insulin” refers to a pro-insulin molecule additionally comprising an N-terminal signal sequence that directs translation to occur on the ER ribosomes.

The term “insulin analog” as used herein is a protein that exhibits insulin activity of the formula AB wherein: A is insulin chain A (alpha) or a functional derivative of chain A of insulin; and B is the insulin chain B (beta) or a functional derivative of the insulin chain B which has an .epsilon-amino group and at least one of A or B contains an amino acid modification of the natural sequence. Table 1 shows the sequences of A- and B-chain of exemplary insulin analogs.

In the present disclosure, whenever the term insulin is used in a plural or a generic sense, it is intended to encompass both naturally occurring insulins and insulin analogs and derivatives thereof.

By “insulin polypeptide” as used herein, it is meant a compound that has a molecular structure similar to that of human insulin, including the disulfide bridges between Cys.sup.A7 and Cys.sup.B7 and between Cys.sup.A20 and Cys.sup.B19 and an internal disulfide bridge between Cys.sup.A6 and Cys.sup.A11, and which have insulin activity.

Insulin peptides include, but are not limited to, insulin, human; insulin, porcine; IGF-1, human; insulin-like growth factor II (69-84); pro-insulin-like growth factor II (68-102), human; insulin-like growth factor II (105-128), human; [AspB28] insulin, human; [LysB28] insulin, human; [LeuB28] insulin, human; [ValB28] insulin, human; [AlaB28] insulin, human; [AspB28, ProB29] insulin, human; [LysB28, ProB29] insulin, human; [LeuB28, ProB29] insulin, human; [ValB28, ProB29] insulin, human; [AlaB28, ProB29] insulin, human; [GlyA21] insulin, human; [GlyA21 GlnB3] insulin, human; [AlaA21] insulin, human; [AlaA21 Gln.sup.B3] insulin, human; [GlnB3] insulin, human; [GlnB30] insulin, human; [GlyA21 GluB30] insulin, human; [GlyA21 GlnB3 GluB30] insulin, human; [GlnB3 GluB30] insulin, human; B22B30 insulin, human; B23B30 insulin, human; B25B30 insulin, human; B26B30 insulin, human; B27B30 insulin, human; B29B30 insulin, human; the A chain of human insulin, and the B chain of human insulin.

As used herein the terms “precursor peptide for human insulin analog” and “modified pro-insulin polypeptide”, which may be used interchangeable herein, refer to precursor peptides for any and all insulin polypeptides, including the insulin polypeptides and/or peptides listed in Table 1 as well as a polypeptide molecule comprising a sequence of amino acid residues which is (i) substantially identical to the amino acid sequences constituting any insulin polypeptides and/or insulin peptide set forth herein or (ii) encoded by a nucleic acid sequence capable of hybridizing under at least moderately stringent conditions to any nucleic acid sequence encoding insulin set forth herein or capable of hybridizing under at least moderately stringent conditions to any nucleic acid sequence encoding insulin set forth herein but for the use of synonymous codons. The terms insulin and insulin polypeptide include pro-insulin polypeptides and mini-insulin polypeptides, analogs and derivatives thereof. The insulin analog polypeptide is preferably of human origin.

The present disclosure contemplates certain derivations or further substitutions of the insulin analogs. It is accordingly possible to derivate one or more of the functional groups in the amino acid residues. Examples of such derivation is per se known conversion of acid groups in the insulin molecule into ester or amid groups, conversion of alcohol groups into alkoxy groups or vice versa, and selective deamidation. As an example, A21Asn may be deamidated into A21Asp by hydrolysis in acid medium or B3Asn may be deamidated into B3Asp in neutral medium.

It is furthermore possible to modify the present insulin analogs by either adding or removing amino acid residues at the N- or C-terminal ends. The insulin analogs of the present disclosure may lack up to four amino acid residues at the N-terminal end of the B-chain and up to five amino acid residues at the C-terminal end of the B-chain without significant impact on the overall properties of the insulin analog. Examples of such modified insulin analogs are insulin analogs lacking the B1Phe or the B30Thr amino acid residue.

Also, naturally occurring amino acid residues may be added at one or more ends of the polypeptide chains provided that this has no significant influence on the overall properties and effects of insulin.

Such deletions or additions at the ends of the polypeptide chain of the present insulin analogs may be exercised in vitro on the insulin analogs with amino acid substitutions according to the present disclosure. Alternatively, the gene for the insulin analogs according to the present disclosure may be modified by either adding or removing codons corresponding to the extra amino acid residues or lacking amino acid residues at the ends of the polypeptide chain, respectively.

The term “modified polynucleotide” herein refers to a polynucleotide sequence that has been altered to contain at least one mutation to encode a “modified” protein. In some instances, the term “polynucleotide” is used without association with “modified”, which does not exclude the embodiments of polynucleotides that are modified.

As used herein, the terms “protease” and “proteolytic activity” refer to a protein or peptide exhibiting the ability to hydrolyze peptides or substrates having peptide linkages.

The term “oligonucleotide” is used for a nucleic acid molecule, DNA (or RNA), with less than 100 nucleotides in length.

“Transformation” means introducing DNA into an organism, i.e. a host organism, so that the DNA is replicable, either as an extrachromosomal element or by chromosomal integration.

Depending on the host cell used, transformation is done using standard techniques appropriate to such cells. General aspects of mammalian cell host system transformations have been described by Axel in U.S. Pat. No. 4,399,216 issued Aug. 16, 1983.

“Cells” or “cell cultures” or “recombinant host cells” or “host cells” are often used interchangeably as will be clear from the context. These terms include the immediate subject cell, which expresses the desired protein of the present invention, and, of course, the progeny thereof. It is understood that not all progeny are exactly identical to the parental cell, due to chance mutations or difference in environment. However, such altered progeny are included in these terms, so long as the progeny retain the characteristics relevant to those conferred on the originally transformed cell.

The term “expression” and the verb “to express” denote transcription of DNA sequences and/or the translation of the transcribed mRNA in a host organism resulting in a pre-protein, i.e. not including post-translational processes.

As used herein, the term “vector” refers to a nucleic acid molecule capable of transporting another nucleic acid to which it has been linked. The term “expression vector” includes plasmids, cosmids or phages capable of synthesizing the subject proteins encoded by their respective recombinant genes carried by the vector. Preferred vectors are those capable of autonomous replication and/expression of nucleic acids to which they are linked. In the present specification, “plasmid” and “vector” are used interchangeably as the plasmid is the most commonly used form of vector. Moreover, the invention is intended to include such other forms of expression vectors which serve equivalent functions and which become known in the art subsequently hereto.

A “promoter” is a regulatory nucleotide sequence that stimulates transcription. These terms are understood by those of skill in the art of genetic engineering. Like a promoter, a “promoter element” stimulates transcription but constitutes a sub-fragment of a larger promoter sequence.

The term “operably linked” refers to the association of two or more nucleic acid fragments on a single vector so that the function of one is affected by the other. For example, a promoter is operably linked with a coding sequence, i.e. a nucleotide sequence encoding a protein or a pre-protein, when it is capable of affecting the expression of that coding sequence, i.e., that the coding sequence is under the transcriptional control of the promoter.

The term “post-translational processing” or “post-translational modification” denotes the modification steps a pre-protein or a pre-pro protein is subjected to, in order result in a mature protein in a cellular or extracellular compartment.

A “signal peptide” is a cleavable signal sequence of amino acids present in the pre-protein or a pre-pro-protein form of a secretable protein. Proteins transported across the cell membrane, i.e. “secreted”, typically have an N-terminal sequence rich in hydrophobic amino acids, typically about 15 to 30 amino acids long. Sometime during the process of passing through the membrane, the signal sequence is cleaved by a signal peptidase (Alberts, B., Johnson, A., Lewis, J., Raff, M., Roberts, K., Walter, P. (eds), Molecular Biology of the Cell, fourth edition, 2002, Garland Science Publishing). Many sources of signal peptides are well-known to those skilled in the art and can include, for example, the amino acid sequence of the α-factor signal peptide from Saccharomyces cerevisiae and the like. In general, the pre-protein N-terminus of essentially any secreted protein is a potential source of a signal peptide suitable for use in the present disclosure. A signal peptide can also be bipartite comprising two signal peptides directing the pre-protein to a first and a second cellular compartment. Bipartite signal peptides are cleaved off stepwise during the course of the secretory pathway.

Pre-proteins with an N-terminal signal peptide are directed to enter the “secretory pathway”. The secretory pathway comprises the processes of post-translational processing and finally results in secretion of a protein. Glycosylation and the formation of disulfide bonds are processes that are part of the secretory pathway prior to secretion.

By the term “substantially identical” it is meant that two polypeptide sequences preferably are at least 75% identical, and more preferably are at least 85% identical and most preferably at least 95% identical, for example 96%, 97%, 98% or 99% identical. In order to determine the percentage of identity between two polypeptide sequences the amino acid sequences of such two sequences are aligned, preferably using the Clustal W algorithm (Thompson, J D, Higgins D G, Gibson T J, 1994, Nucleic Adds Res. 22 (22): 4673-4680, together with BLOSUM 62 scoring matrix (Henikoff S. and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Adds Res., 1984, 12: 387) BLASTP, BLASTN and FASTA (Altschul et al., J. Molec. Biol., 1990:215:403).

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log 10 [Na+])+0.41(% (G+C)−600/1), or similar equation). Accordingly, the parameters in the wash conditions that determine hybrid stability are sodium ion concentration and temperature.. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

TABLE 1
Insulin Analogs

| | Alpha | | Disulfide |
|---|---|---|---|
| Name | chain | Beta chain | bound |
| Lispro | GIVEQCCTSIC | FVNQHLCGSH | Alpha 6-alpha |
| | SLYQLENYCN | LVEALYLVCG | 11; |
| | (SEQ ID | ERGFFYTKPT | Alpha 7-beta 7; |
| | NO: 10) | (SEQ ID | Alpha 20-beta |
| | | NO: 11) | 19 |
| Aspart | GIVEQCCTSIC | FVNQHLCGS | Alpha 6-alpha |
| | SLYQLENYCN | HLVEALYLV | 11; |
| | (SEQ ID | CGERGFFYTD | Alpha 7-beta 7; |
| | NO: 12) | KT (SEQ ID | Alpha 20-beta |
| | | NO: 13) | 19 |
| Glargine | GIVEQCCTSIC | FVNQHLCGS | Alpha 6-alpha |
| | SLYQLENYCG | HLVEALYLV | 11; |
| | (SEQ ID | CGERGFFYT | Alpha 7-beta 7; |
| | NO: 14) | PKTRR | Alpha 20-beta |
| | | (SEQ ID | 19 |
| | | NO: 15) | |

The term “chimeric” as used herein in the context of nucleic add sequences refers to at least two linked nucleic add sequences which are not naturally linked. Chimeric nucleic add sequences include linked nucleic acid sequences of different natural origins. Chimeric nucleic acid sequences also may comprise nucleic add sequences of the same natural origin, provided they are not naturally linked. For example a nucleic acid sequence constituting a promoter obtained from a particular cell-type may be linked to a nucleic add sequence encoding a polypeptide obtained from that same cell-type, but not normally linked to the nucleic acid sequence constituting the promoter. Chimeric nucleic acid sequences also include nucleic acid sequences comprising any naturally occurring nucleic add sequence linked to any non-naturally occurring nucleic add sequence.

Disclosed herein are novel methods for the recombinant production of insulin and/or insulin analogs or derivatives thereof in mammalian cells. The methods described herein describe a specific combination of conditions and reagents that result in increased recombinant insulin and/or insulin analog expression as a soluble protein in a host cell and do not require extraction from inclusion bodies or an in vitro refolding or disulfide-shuffling step. One advantage conferred by the methods disclosed herein is that due to the increased production of properly folded and processed insulin and/or insulin analog protein in the soluble fraction of the host cell, shorter, and simplified protein extraction and purification schemes may be utilized.

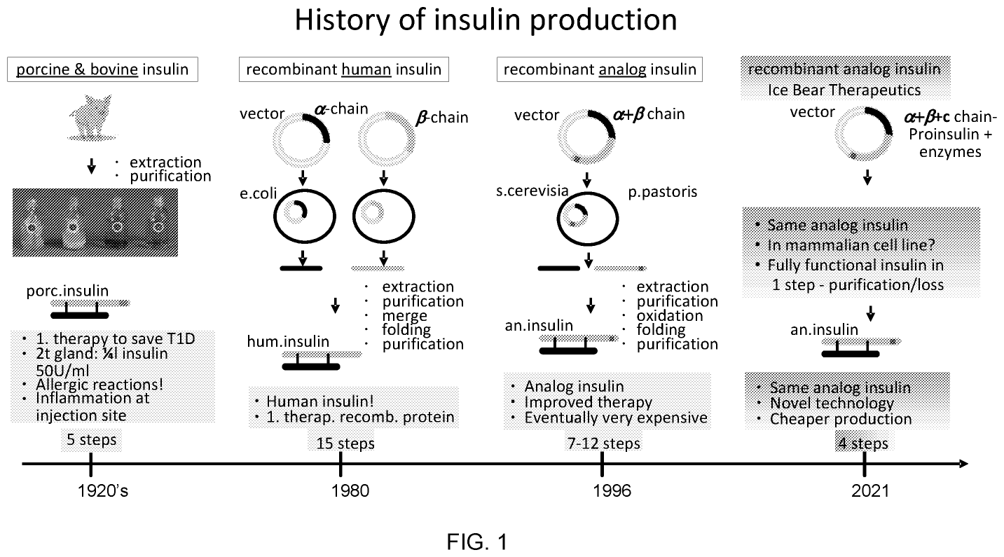

FIG. 1 depicts the history of insulin production and further provides a schematic overview of the four-step synthesis strategy of insulin analogs by the methods of the present disclosure. Recombinant human insulin first entered clinical trials in humans in 1980. At that time, the A and B chains of the insulin molecule were produced separately in Escherichia coli and then combined by chemical techniques (Frank et al., 1981). Since 1986, a different recombinant process has been used. The human genetic coding for pro-insulin including the A and B chains is inserted into Saccharomyces cerevisiae (Thim et al., 1986) or Pichia pastoris (Wang et al, 2001). The connecting peptide is cleaved enzymatically from pro-insulin to form human insulin. However, all these procedures suffer from lower yields and higher costs than desired. The present disclosure relates to improved procedures for the production of insulin in mammalian cells. According to the present disclosure, the A chain, B chain and C peptide are co-expressed in a vector in mammalian cells to enable proper folding and secretion of insulin from the cell. The A and B chains make up the insulin molecule and the C peptide is a connecting peptide that holds the two chains together. Inclusion of the C peptide of proinsulin in the vector expression system may ensure efficient and fast reading and translation of amino acid sequence, proper folding and stability of the insulin molecule, reducing protein loss and improving insulin output. When the proinsulin is cleaved by proteases, the A and B chains are separated from the C peptide, allowing insulin to be formed and released. Addition of proteases, such as PC1/3, PC2 and/or CPE, in the cell by co-expressing in the same vector or in a separate vector ensures successful cutting and/or trimming to produce fully functional insulin. In some aspects, a vector encoding a pro-insulin including A and B chains and C peptide as well as one or more agents that modify pro-insulin to functional insulin is introduced into a mammalian cell line. In another aspect, two vectors, one encoding a pro-insulin including A and B chains and C peptide and the other encoding one or more agents that modify pro-insulin to functional insulin, are co-introduced into a mammalian cell line. Fully functional insulin is secreted by the cells and harvested without lysing the cells.

The methods of the present application are an important advantage over the prior art. The insulin producing cells of the present disclosure are capable of continuously secreting functional insulin. Continuous secretion of fully functional insulin permits immediate capture and purification, enabling a closed loop system allowing the process to be fully automated. In accordance with one exemplary non-limiting embodiment, an integrated unit comprising a perfusion bioreactor and multicolumn chromatography with a continuous flow of material throughout is provided for maintaining insulin-producing cells and capturing the secreted insulin. The bioreactor is perfused at a set rate and its effluent fed directly into the purification train, leading to periodic or continuous insulin collection over a similar or extended time frame to meet product demands. Details of continuous manufacturing processes are described in Chiang, et al, Biotechnology and Bioengineering. 2019 and Lute et al, Biotechnol Progress. 2020, the contents of each of which are hereby expressly incorporated by reference in their entirety for any purpose. The insulin production procedure of the present disclosure is a continuous process as opposed to the intermittent production process of the current technology. Continuous manufacturing allows for shorter processing times, reduced manual intervention, simple and quick adjustment of output in response to demand, and a reduction in facility size through the use of smaller or fewer tanks, bioreactors, and columns. For example, the size of a facility may be reduced to the size of a living room, as opposed to a huge factory hall, due to one tank instead of multiple tanks being used. The present technology significantly shortens insulin production time from months to 1-4 weeks.

In certain aspects, the present disclosure provides recombinant nucleic acids encoding insulin analogs, expression vectors containing the insulin analogs, host cells comprising the variant nucleic acids and/or expression vectors, and methods for producing the variant proteins.

Thus the present disclosure relates to processes or methods of producing insulin and/or insulin analogs in a mammalian host cell that avoid the expensive and time consuming downstream purification and processing steps to provide a higher yield of insulin and/or insulin analogs, thereby driving costs down to make insulin, insulin analogs and/or derivatives thereof more affordable.

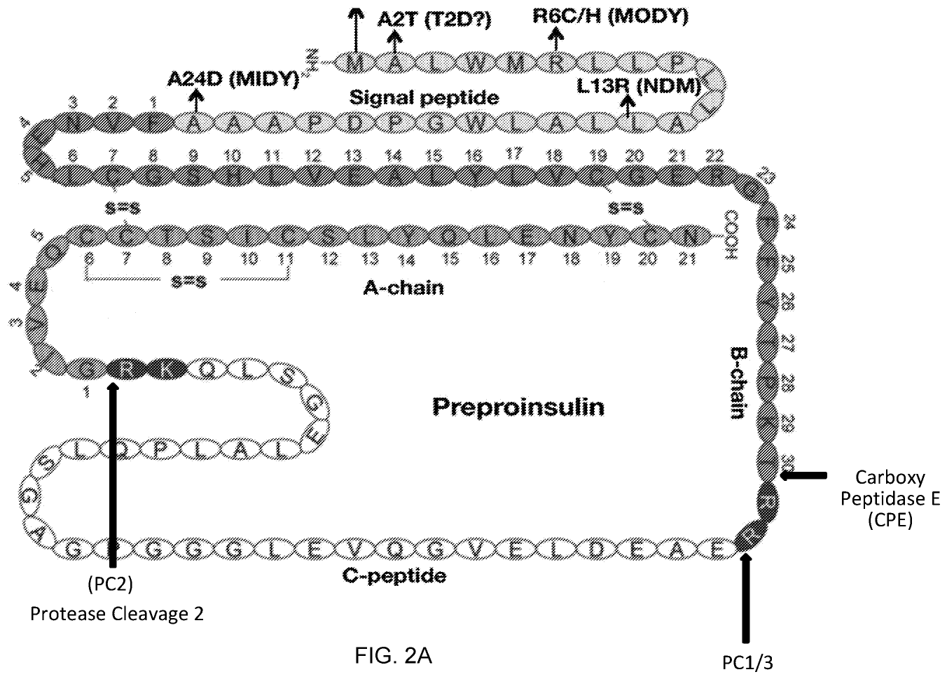

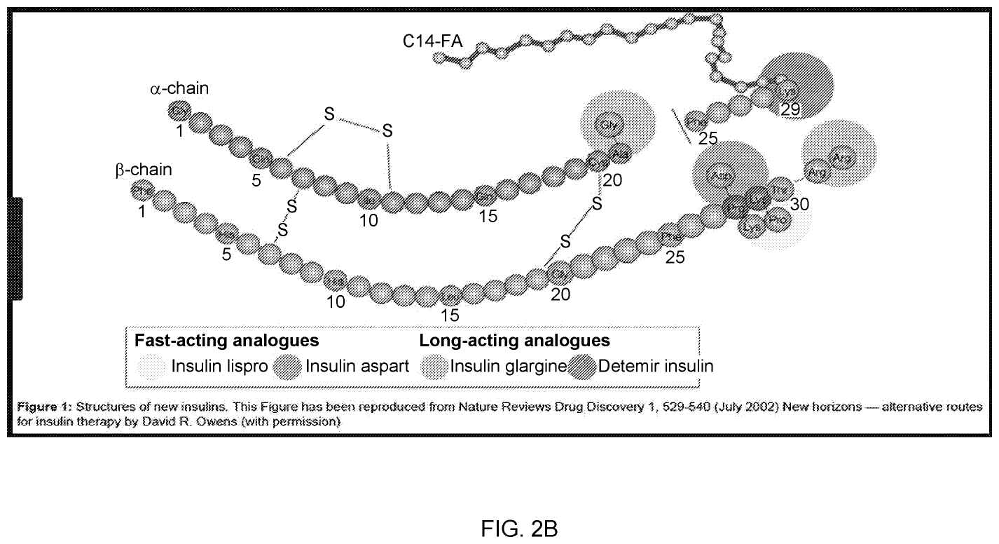

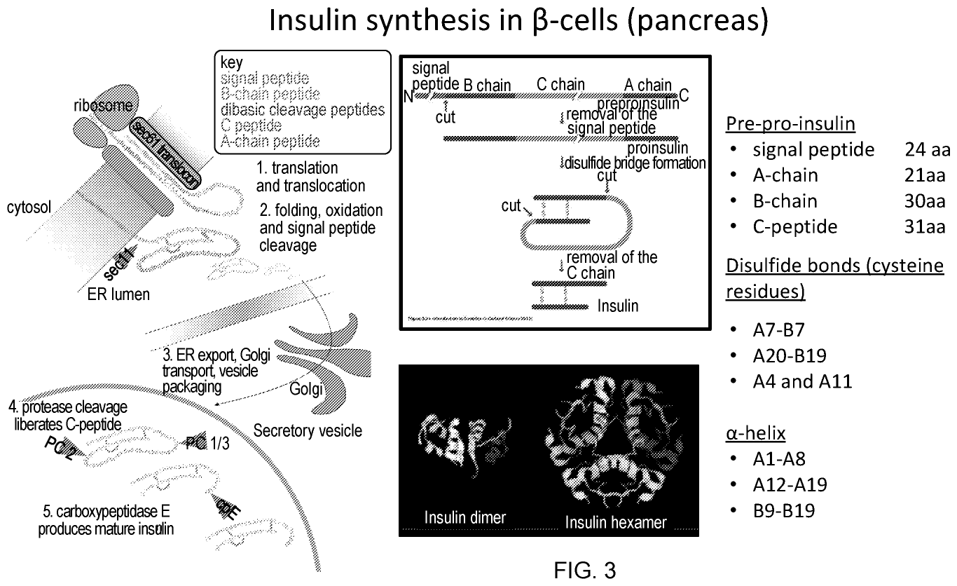

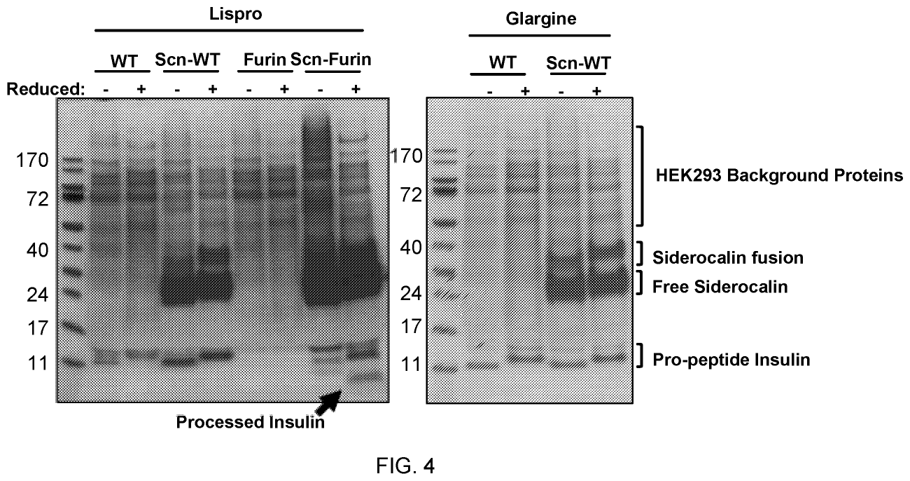

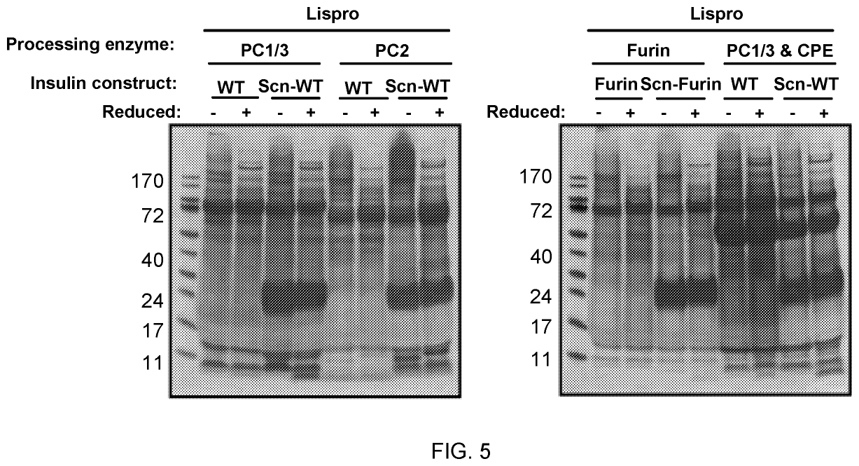

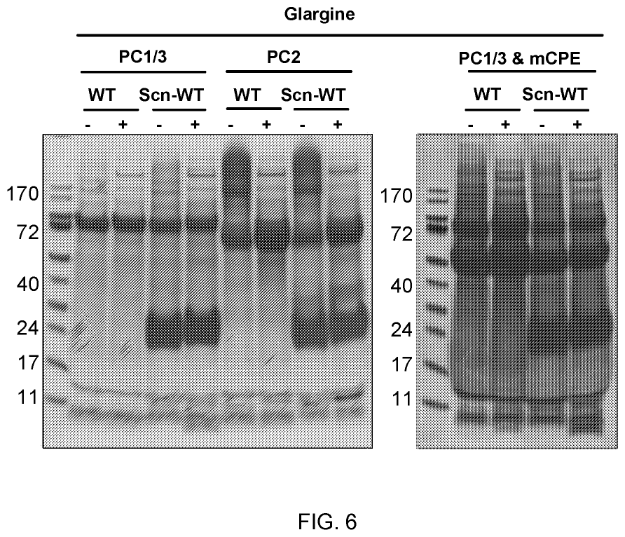

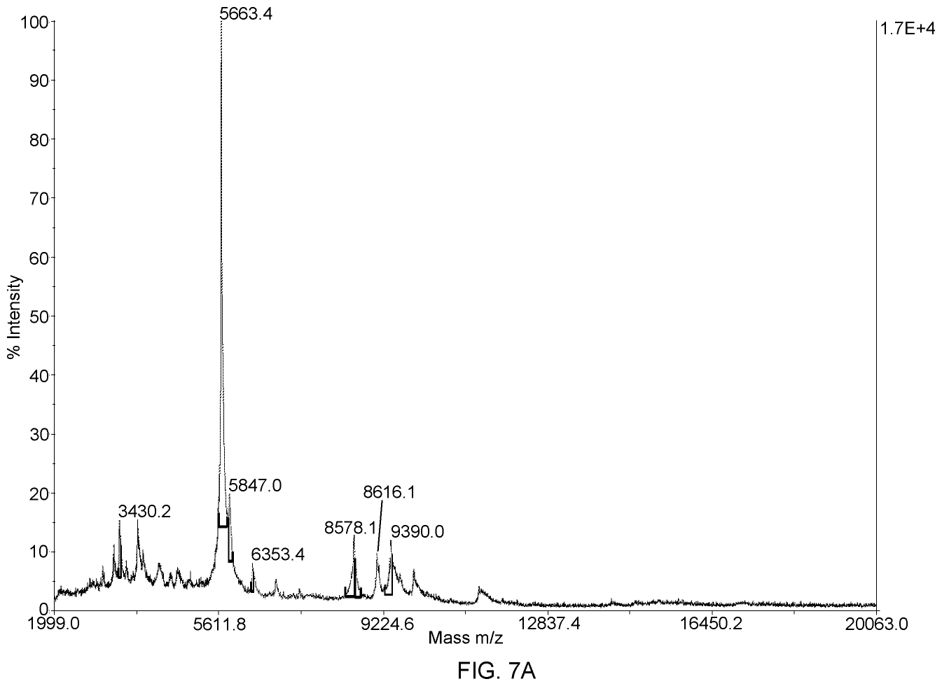

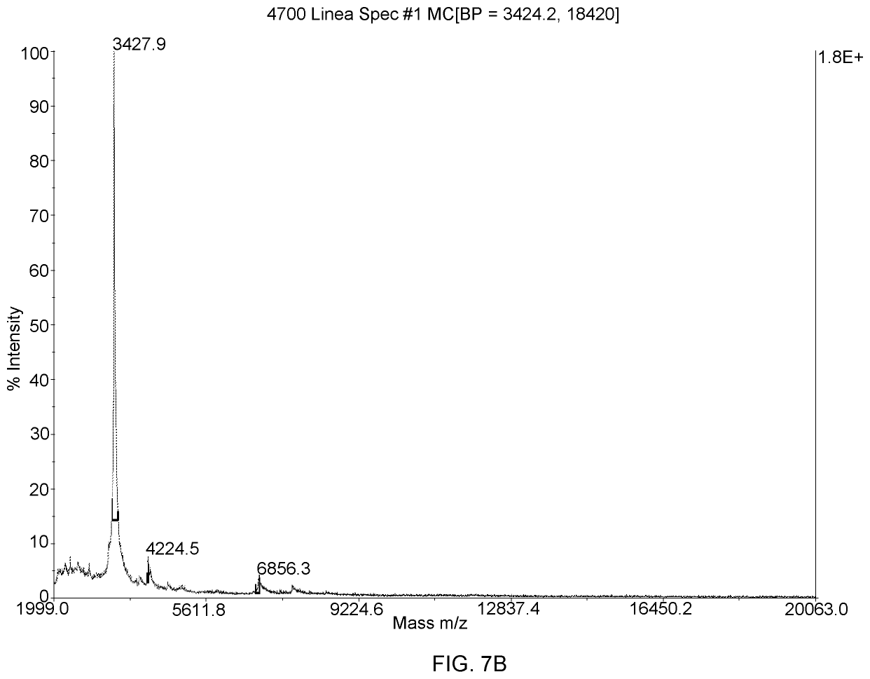

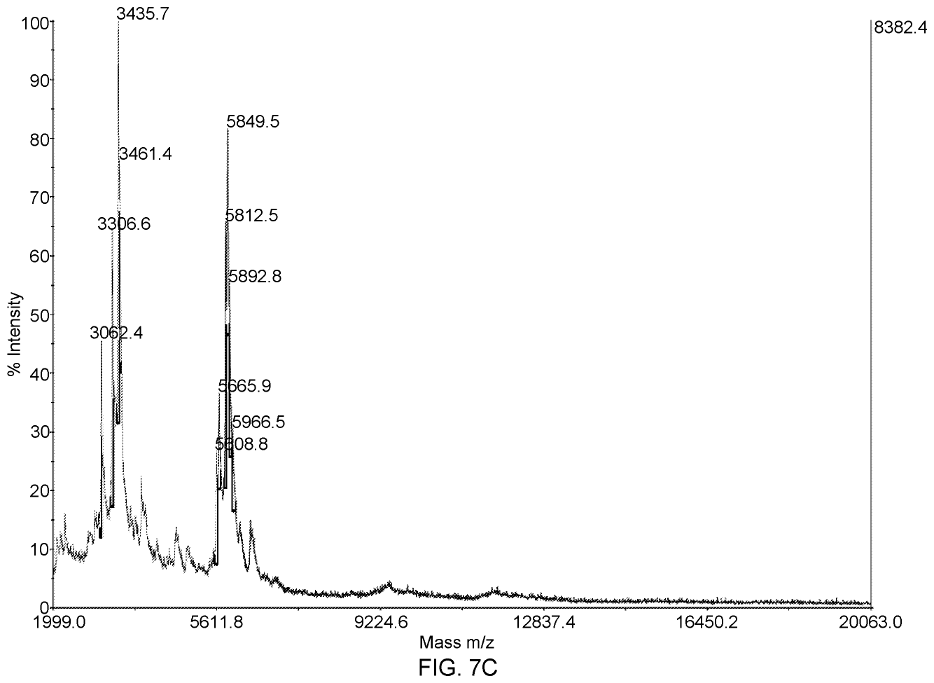

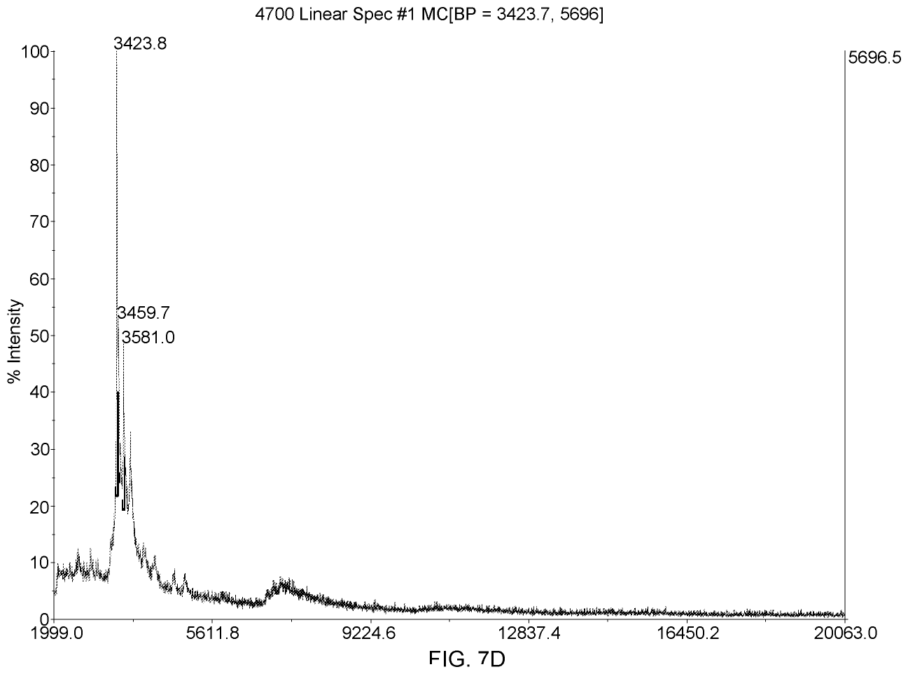

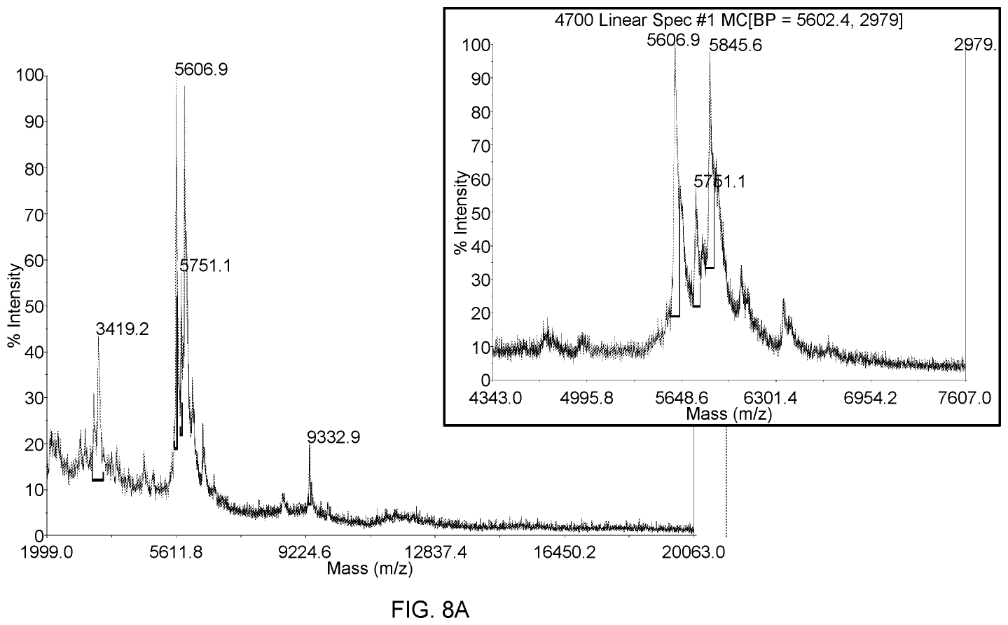

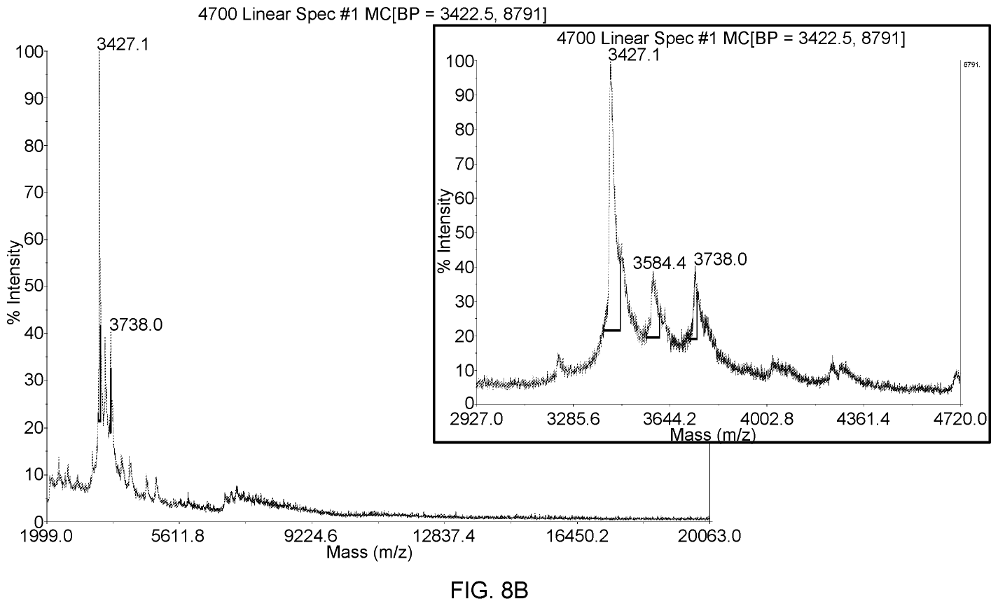

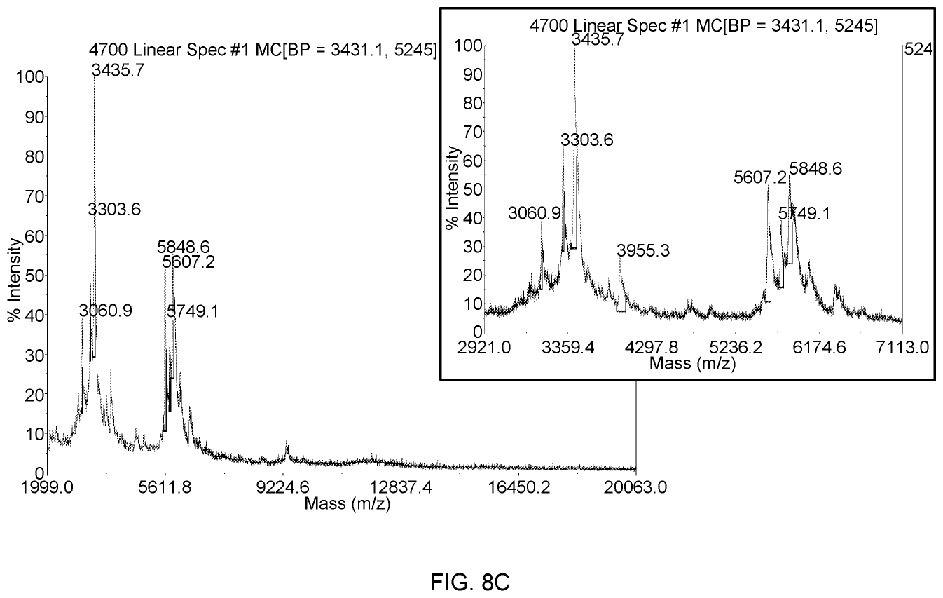

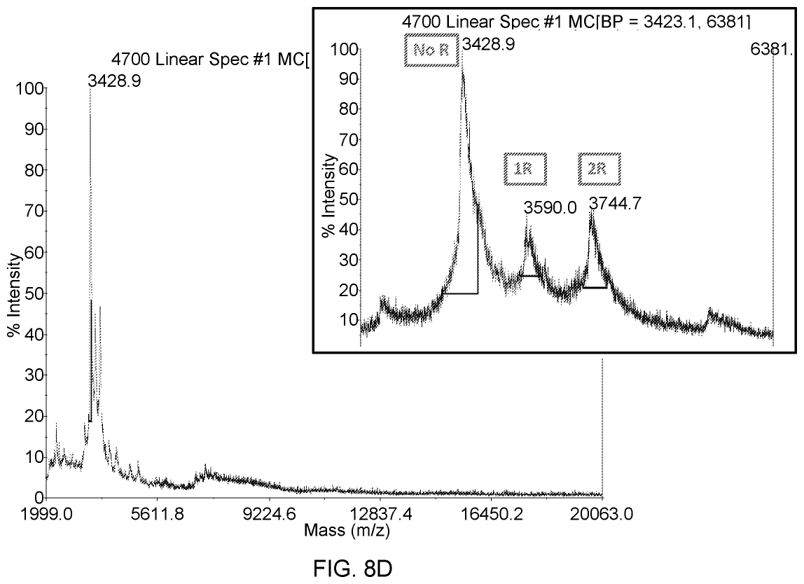

The biosynthesis of insulin via its precursors, prepro-insulin and pro-insulin, is one of the key processes that ensures the production of sufficient amounts of insulin in the pancreatic β cell. See Steiner D., Chan, S. & Rubenstein, A. (2000) in Handbook of Physiology, The Endocrine System, eds. Jefferson, L. & Cherrington, A. (Oxford Univ. Press, New York), Vol. II, pp. 49-77, Goodge K. A. & Hutton, J. C. (2000) Semin. Cell. Dev. Biol. 11, 235-242. The efficient conversion of pro-insulin to insulin requires cleavages at both junctions of the connecting segment linking the B and A chains to release insulin and C-peptide. These products normally are stored within the mature secretory granules (>95% of total insulin-related material) awaiting secretion in response to glucose and other stimuli. As shown in FIG. 2A, initial processing cleavages occur between residues 32 and 33 (R↓E) and residues 65 and 66 (R↓G) at the B and A chain junctions, respectively. Recognition of each of these sites by the neuroendocrine convertases PC1/3 (SPC3) and PC2 (SPC2) (Seidah N. G. & Chrétien, M. (1999) Brain Res. 848, 45-62, Zhou A., Webb, G., Zhu, X. & Steiner, D. F. (1999) J. Biol. Chem. 274, 20745-20748) involves interactions with 4-6 residues upstream and at least two residues downstream, i.e., in human pro-insulin resides 27-34 and 60-67, surrounding these two sites. The initial cleavage products, consisting of insulin extended at the B chain C terminus by R³¹-R³² and C-peptide extended C-terminally by K⁶⁴-R⁶⁵, are then trimmed by removal of these basic residues by carboxypeptidase E (CPE) (L. Fricker L. (1988) Annu. Rev. Physiol. 50, 309-321.pmid:2897826) to yield the mature β cell secretory products. These convertases and CPE act mainly within maturing dense-core granules in the regulated secretory pathway.

FIG. 2B illustrates the structures of several insulin analogs. Insulin lispro and insulin aspart are rapid-acting analogs that have reduced self-association as a result of protein engineering. In insulin lispro, a lysine-proline (Lys-Pro) sequence at the end of the insulin-B chain is reversed, which creates steric hindrance and a reduced ability to self-associate. Insulin aspart incorporates an amino-acid change (Pro B28 to aspartic acid Asp) that also creates charge repulsion and steric hindrance due to a local conformational change at the carboxyl terminus of the B chain. Insulin glargine is a long-acting insulin that contains two extra arginine molecules at the end of the B chain (Arg B31 and Arg B32) to alter the isoelectric point. A glycine substitution at A21 (A chain) was made to stabilize the molecule. Insulin detemir is another long-acting insulin that contains acylation of the F-amino group of Lys B29. Acylation promotes reversible binding to insulin to albumin, thereby delaying its absorption from the subcutaneous tissue and transport across the capillary endothelium of skeletal muscle.

Earlier methods of manufacturing insulin produced the A and B chains by transfecting appropriate host cells with vectors encoding the A and the B chains separately. The two chains are then purified and then mixed together and joined by disulfide bonds through the reduction-reoxidation reaction, followed by purification. The purification step is accomplished by chromatography, or separation, techniques that exploit differences in the molecule's charge, size, and affinity to water. Procedures used include an ion-exchange column, reverse-phase high performance liquid chromatography, and a gel filtration chromatography column. Alternatively, biosynthetic human insulin and analogs thereof are produced by the transformation of a single chain fusion protein. The method of producing these drugs is generally described in numerous patent specifications and scientific publications and is based on the overexpression of a gene encoding prepro-insulin, i.e. a hybrid polypeptide consisting of a leader protein and pro-insulin, i.e. the insulin B-chain (or derivative thereof), a linker peptide and the insulin A-chain (or derivative thereof). Once the fusion protein is isolated, the next manufacturing step is removal of the leader protein and the linker peptide, followed by isolation of the pure hormone.

In certain aspects, the present disclosure provides a method for the production of recombinant functional human insulin analog or derivatives thereof by culturing a host cell comprising a heterologous nucleic acid sequence encoding a precursor peptide or a modified proinsulin peptide for human insulin analog and/or derivatives thereof. In some aspect, the nucleic acid sequence encoding the precursor peptide for human insulin analog or derivatives thereof further encodes one or more agents for processing the modified proinsulin or derivatives thereof to functional insulin analog or derivatives thereof. In some aspects, the precursor of the human insulin analog or derivatives thereof comprises the B-chain of human insulin or an analog thereof, the A-chain of human insulin or an analog thereof and a C-peptide linking the B-chain and the A-chain together.

Preferred heterologous nucleic acids encode a pro-insulin polypeptide. In certain aspects the heterologous nucleic acid sequence encodes a modified pro-insulin polypeptide that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 1, SEQ ID: No. 2; SEQ ID: No. 3, and SEQ ID: No. 4. (Table 2) In some aspects, the heterologous nucleic acid encodes a polypeptide that has an amino acid sequence as set forth in: SEQ ID:1, SEQ ID:2; SEQ ID:3, and SEQ ID: No. 4. In some aspects, the modified pro-insulin polypeptide comprises an insulin chain A, an insulin chain B, and a connecting C peptide connecting chain A and chain B. In some aspects, the insulin chain A comprises the sequence that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID NO: 9, 11 or 13. In some aspects, the insulin chain B comprises the sequence that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID NO: 11, 13 or 15.

TABLE 2
Pro-insulin polypeptide constructs for insulin analog

| SEQ | INSULIN | |
|---|---|---|
| ID NO. | ANALOG | SEQUENCE |
| 1 | Lispro | MALWMRLLPLLALLALWGPDPAAA/FVNQHLCGSHLVEALYL |
| | | VCGERGFFYTKPTRR/EAEDLQVGQVELGGGPGAGSLQPLAL |
| | | EGSLQKR/GIVEQCCTSICSLYQLENYCN |
| 2 | Lispro SCN | METDTLLLWVLLLWVPGSTG/DYKDEHHHHHHGGSQDSTSDLIP |
| | Fusion | APPLSKVPLQQNFQDNQFQGKWYVVGLAGNAILREDKDPQKMYA |
| | | TIYELKEDKSYNVTSVLFRKKKCDYWIRTFVPGSQPGEFTLGNI |
| | | KSYPGLTSYLVRVVSTNYNQHAMVFFKKVSQNREYFKITLYGRT |
| | | KELTSELKENFIRFSKSLGLPENHIVFPVPIDQCIDGGGSRRKR/ |
| | | FVNQHLCGSHLVEALYLVCGERGFFYTKPTRR/EAEDLQVGQVE |
| | | LGGGPGAGSLQPLALEGSLQKR/GIVEQCCTSICSLYQLENYCN |
| 3 | Glargine | MALWMRLLPLLALLALWGPDPAAA/FVNQHLCGSHLVEALYLV |
| | | CGERGFFYTPKTRR/EAEDLQVGQVELGGGPGAGSLQPLALEG |
| | | SLQKR/GIVEQCCTSICSLYQLENYCG |
| 4 | Glargine | METDTLLLWVLLLWVPGSTG/DYKDEHHHHHHGGSQDSTSDLIP |
| | SCN Fusion | APPLSKVPLQQNFQDNQFQGKWYVVGLAGNAILREDKDPQKMYA |
| | | TIYELKEDKSYNVTSVLFRKKKCDYWIRTFVPGSQPGEFTLGNI |
| | | KSYPGLTSYLVRVVSTNYNQHAMVFFKKVSQNREYFKITLYGRT |
| | | KELTSELKENFIRFSKSLGLPENHIVFPVPIDQCIDGGGSRRKR/ |
| | | FVNQHLCGSHLVEALYLVCGERGFFYTPKTRR/EAEDLQVGQVE |
| | | LGGGPGAGSLQPLALEGSLQKR/GIVEQCCTSICSLYQLENYCG |

Note:
signal peptide sequences are underlined; B chain sequences are bolded; C peptide sequences are italic; A chain sequences are bolded and italic.

In certain aspects, the one or more agents for processing the modified pro-insulin to functional insulin analog or derivatives thereof comprise one or more endoproteases and carboxypeptidase E. In some aspects, the endoproteases comprise the Prohormone Convertase 1/3 (PC1/3) and Prohormone Convertase 2 (PC2). The carboxypeptidase enzyme may be any carboxypeptidase enzyme capable of efficient removal of the C-terminal extension of the B-chain. A well suited enzyme is the carboxypeptidase Y enzyme (CPY) or a mutant variant thereof. In certain aspects, the mammalian host cell is modified to express the one or more agents for processing the modified pro-insulin to functional insulin analog or derivatives thereof. In some aspects, the one or more agents has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 5, SEQ ID: No. 6; SEQ ID: No. 7, and SEQ ID: No. 8. In certain aspects, the one or more agents has an amino acid sequence as set forth in SEQ ID: No. 5, SEQ ID: No. 6; SEQ ID: No. 7, and SEQ ID: No. 8.

TABLE 3
Agents for processing pro-insulin to mature insulin

| SEQ | AGENT FOR | |
|---|---|---|
| ID | PROCESSING | |
| NO. | PRO-INSULIN | SEQUENCE |
| 5 | PC1/3 Preproprotein | MERRAWSLQCTAFVLFCAWCALNSAKAKRQFVNEWAAEIPGG |
| | | PEAASAIAEELGYDLLGQIGSLENHYLFKHKNHPRRSRRSAF |
| | | HITKRLSDDDRVIWAEQQYEKERSKRSALRDSALNLFNDPMW |
| | | NQQWYLQDTRMTAALPKLDLHVIPVWQKGITGKGVVITVLDD |
| | | GLEWNHTDIYANYDPEASYDFNDNDHDPFPRYDPTNENKHGT |
| | | RCAGEIAMQANNHKCGVGVAYNSKVGGIRMLDGIVTDAIEA |
| | | SSIGFNPGHVDIYSASWGPNDDGKTVEGPGRLAQKAFEY |
| | | GVKQGRQGKGSIFVWASGNGGRQGDNCDCDGYTDSIYTIS |
| | | ISSASQQGLSPWYAEKCSSTLATSYSSGDYTDQRITSADL |
| | | HNDCTETHTGTSASAPLAAGIFALALEANPNLTWRDMQHL |
| | | VVWTSEYDPLANNPGWKKNGAGLMVNSRFGFGLLNAKAL |
| | | VDLADPRTWRSVPEKKECVVKDNDFEPRALKANGEVIIEIP |
| | | TRACEGQENAIKSLEHVQFEATIEYSRRGDLHVTLTSAAGT |
| | | STVLLAERERDTSPNGFKNWDFMSVHTWGENPIGTWTLRI |
| | | TDMSGRIQNEGRIVNWKLILHGTSSQPEHMKQPRVYTSYN |
| | | TVQNDRRGVEKMVDPGEEQPTQENPKENTLVSKSPSSSSV |
| | | GGRRDELEEGAPSQAMLRLLQSAFSKNSPPKQSPKKSPSAK |
| | | LNIPYENFYEALEKLNKPSQLKDSEDSLYNDYVDVFYNTKP |
| | | YKHRDDRLLQALVDILNEEN |
| 6 | PC2 Preproprotein | MKGGCVSQWKAAAGFLFCVMVFASAERPVFTNHFLVELHKG |
| | | GEDKARQVAAEHGFGVRKLPFAEGLYHFYHNGLAKAKRRRS |
| | | LHHKQQLERDPRVKMALQQEGFDRKKRGYRDINEIDINMND |
| | | PLFTKQWYLINTGQADGTPGLDLNVAEAWELGYTGKGVTIG |
| | | IMDDGIDYLHPDLASNYNAEASYDFSSNDPYPYPRYTDDWE |
| | | NSHGTRCAGEVSAAANNNICGVGVAYNSKVAGIRMLDQPFM |
| | | TDIIEASSISHMPQLIDIYSASWGPTDNGKTVDGPRELTLQ |
| | | AMADGVNKGRGGKGSIYVWASGDGGSYDDCNCDGYASSMW |
| | | TISINSAINDGRTALYDESCSSTLASTFSNGRKRNPEAGV |
| | | ATTDLYGNCTLRHSGTSAAAPEAAGVFALALEANLGLTWR |
| | | DMQHLTVLTSKRNQLHDEVHQWRRNGVGLEFNHLFGYGV |
| | | LDAGAMVKMAKDWKTVPERFHCVGGSVQDPEKIPSTGKL |
| | | VLTLTTDACEGKENFVRYLEHVQAVITVNATRRGDLNIN |
| | | MTSPMGTKSILLSRRPRDDDSKVGFDKWPFMTTHTWGED |
| | | ARGTWTLELGFVGSAPQKGVLKEWTLMLHGTQSAPYIDQ |
| | | VVRDYQSKLAMSKKEELEEELDEAVERSLKSILNKN |
| 7 | Pro- | MAGRGGSALLALCGALAACGWLLGAEAQEPGAPAAGMRRR |
| | Carboxypeptidase E | RRLQQEDGISFEYHRYPELREALVSVWLQCTAISRIYTVGR |
| | | SFEGRELLVIELSDNPGVHEPGEPEFKYIGNMHGNEAVGRE |
| | | LLIFLAQYLCNEYQKGNETIVNLIHSTRIHIMPSLNPDGFE |
| | | KAASQPGELKDWFVGRSNAQGIDLNRNFPDLDRIVYVNEKE |
| | | GGPNNHLLKNMKKIVDQNTKLAPETKAVIHWIMDIPFVLSA |
| | | NLHGGDLVANYPYDETRSGSAHEYSSSPDDAIFQSLARAYS |
| | | SFNPAMSDPNRPPCRKNDDDSSFVDGTTNGGAWYSVPGGMQ |
| | | DFNYLSSNCFEITVELSCEKFPPEETLKTYWEDNKNSLISY |
| | | LEQIHRGVKGFVRDLQGNPIANATISVEGIDHDVTSAKDGD |
| | | YWRLLIPGNYKLTASAPGYLAITKKVAVPYSPAAGVDFEL |
| | | ESFSERKEEEKEELMEWWKMMSETLNF |
| 8 | Pro- | MAGRGGSALLALCGALAACGWLLGAEAQEPGAPAAGMRRR |
| | Carboxypeptidase E | RRLQQEDGISFEYHRYPELREALVSVWLQCTAISRIYTVGR |
| | | SFEGRELLVIELSDNPGVHEPGEPEFKYIGNMHGNEAVGRE |
| | | LLIFLAQYLCNEYQKGNETIVNLIHSTRIHIMPSLNPDGFE |
| | | KAASQPGELKDWFVGRSNAQGIDLNRNFPDLDRIVYVNEKE |
| | | GGPNNHLLKNMKKIVDQNTKLAPETKAVIHWIMDIPFVLSA |
| | | NLHGGDLVANYPYDETRSGSAHEYSSSPDDAIFQSLARAYS |
| | | SFNPAMSDPNRPPCRKNDDDSSFVDGTTNGGAWYSVPGG |
| | | MQDFNYLSSNCFEITVALSCEKFPPEETLKTYWEDNKNSLI |
| | | SYLEQIHRGVKGFVRDLQGNPIANATISVEGIDHDVTSAKD |
| | | GDYWRLLIPGNYKLTASAPGYLAITKKVAVPYSPAAGVDF |
| | | ELESFSERKEEEKEELMEWWKMMSETLNF |
| 9 | Soluble | LQQEDGISFEYHRYPELREALVSVWLQCTAISRIYTVGRSF |
| | Carboxypeptidase E | EGRELLVIELSDNPGVHEPGEPEFKYIGNMHGNEAVGRELL |
| | protease | IFLAQYLCNEYQKGNETIVNLIHSTRIHIMPSLNPDGFEKA |
| | | ASQPGELKDWFVGRSNAQGIDLNRNFPDLDRIVYVNEKEG |
| | | GPNNHLLKNMKKIVDQNTKLAPETKAVIHWIMDIPFVLSA |
| | | NLHGGDLVANYPYDETRSGSAHEYSSSPDDAIFQSLARAYS |
| | | SFNPAMSDPNRPPCRKNDDDSSFVDGTTNGGAWYSVPGGMQ |
| | | DFNYLSSNCFEITVELSCEKFPPEETLKTYWEDNKNSLISY |
| | | LEQIHRGVKGFVRDLQGNPIANATISVEGIDHDVTSAKDGD |
| | | YWRLLIPGNYKLTASAPGYLAITKKVAVPYSPAAGVDFELE |
| | | SFSERKEEEKEELMEWWKMMSETLNF |

Note:
the signal peptide sequences are underlined; the pro-peptide sequences are italic; protease sequences are bolded; active site residue is bolded and underlined.

According to the present disclosure, nucleic acids encoding an insulin analog of interest will be isolated, cloned and often altered using recombinant methods. Such aspects are used, including but not limited to, for protein expression or during the generation of variants, derivatives, expression cassettes, or other sequences derived from an insulin polypeptide.

In some aspects, the sequences encoding the polypeptides of the invention are operably linked to a heterologous promoter, for example, a strong constitutive promoter. Strong constitutive promoters which drive expression in many mammalian cell types include, but are not limited to, the adenovirus major late promoter, the human cytomegalovirus immediate early promoter, the SV40 and Rous Sarcoma virus promoter, and the murine 3-phosphoglycerate kinase promoter, EF1α.

The nucleic acid sequences encoding the insulin analog or derivatives thereof that may be used in accordance with the methods and compositions provided herein may be any nucleic acid sequence encoding an insulin analog polypeptide, including any pro-insulin and prepro-insulin and/or their respective analog forms. In certain aspects, the nucleic acid may contain one or more substitutions, additions, deletions, or insertions. Due to redundancy in the genetic code, nucleic acid variants may or may not affect amino acid sequence. Exemplary nucleic add sequences encoding insulin analogs, pro-insulin and prepro-insulin and/or their respective analog forms are well known to the art and are generally readily available from a diverse variety of mammalian sources including human (Bell, G. I. et al., 1980, Nature 284:26-32). Alternative methods to isolate additional nucleic acid sequences encoding insulin polypeptides may be used, and novel sequences may be discovered and used in accordance with the present disclosure. In preferred aspects, nucleic acid sequences encoding insulin analogs or derivatives thereof are human insulin. The nucleic acid sequence encoding the insulin analogs and/or insulin precursor analogs or derivatives thereof may be of genomic or cDNA origin, for instance be obtained by preparing a genomic or cDNA library and screening for nucleic acid sequences coding for all or part of the polypeptide by hybridization using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The nucleic acid sequence encoding the insulin analogs and/or insulin analog precursors or derivatives thereof may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The nucleic acid sequence may also be prepared by polymerase chain reaction using specific primers, for instance as described in U.S. Pat. No. 4,683,202 or Saiki et al., Science 239 (1988), 487-491.

A nucleotide sequence encoding an insulin analog comprising a non-naturally encoded amino acid may be synthesized on the basis of the amino acid sequence of the parent polypeptide, including but not limited to, having the amino acid sequence shown in SEQ ID NO: 1, SEQ ID NO: 2, SEQ ID NO: 3, and SEQ ID NO.: 4, and then changing the nucleotide sequence so as to effect introduction (i.e., incorporation or substitution) or removal (i.e., deletion or substitution) of the relevant amino acid residue(s). The nucleotide sequence may be conveniently modified by site-directed mutagenesis in accordance with conventional methods. Alternatively, the nucleotide sequence may be prepared by chemical synthesis, including but not limited to, by using an oligonucleotide synthesizer, wherein oligonucleotides are designed based on the amino acid sequence of the desired polypeptide, and preferably selecting those codons that are favored in the host cell in which the recombinant polypeptide will be produced.

Numerous insulin analogs are known to the prior art (see for example U.S. Pat. Nos. 5,461,031; 5,474,978; 5,164,366 and 5,008,241) and may be used in accordance with the present disclosure. Analogs that may be used herein include human insulin molecules in which amino add residue 28 of the B-chain (B28) has been changed from its natural proline residue into aspartate, lysine or isoleucine. In certain aspects, the lysine residue at B29 is modified to a proline. Furthermore, the aspargine at A21 may be changed to alanine, glutamine, glutamate, glycine, histidine, isoleucine, leucine, methionine, serine, threonine, tryptophan, tyrosine or valine. Further examples of insulin analogs that may be used herein include: human insulin lacking the B30 residue, also frequently referred to as “desB30” or “B(1-29); those lacking the last 3 amino acid residues insulin “B(1-27)”; insulin molecules lacking the phenylalanine residue at B1; and analogs wherein the A-chain or the B-chain have an N-terminal or C-terminal extension, for example the B-chain may be N-terminally extended by the addition of two arginine residues. The present disclosure utilizes routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

General texts which describe molecular biological techniques include Berger and Kimmel, Guide to Molecular Cloning Techniques, Methods in Enzymology volume 152 Academic Press, Inc., San Diego, Calif (Berger); Sambrook et al., Molecular Cloning A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). These texts describe mutagenesis, the use of vectors, promoters and many other relevant topics related to, including but not limited to, the generation of genes or polynucleotides that include selector codons for production of proteins that include unnatural amino acids, orthogonal tRNAs, orthogonal synthetases, and pairs thereof.

The “operational elements,” as discussed herein, include at least one promoter, at least one operator, at least one leader sequence, at least one Shine-Dalgarno sequence, at least one terminator codon, and any other DNA sequences necessary or preferred for appropriate transcription and subsequent translation of the vector DNA. In particular, it is contemplated that such vectors will contain at least one origin of replication recognized by the host microorganism along with at least one selectable marker and at least one promoter sequence capable of initiating transcription of the synthetic DNA sequence. It is additionally preferred that the vector, in one aspect, contains certain DNA sequences capable of functioning as regulators, and other DNA sequences capable of coding for regulator protein. These regulators, in one aspect, serve to prevent expression of the DNA sequence in the presence of certain environmental conditions and, in the presence of other environmental conditions, allow transcription and subsequent expression of the protein coded for by the DNA sequence.

In accordance herewith, the nucleic acid sequence encoding at least one insulin analog or derivatives thereof is linked to a promoter capable of controlling expression of the insulin polypeptide in a mammalian host cell. Alternatively, the nucleic acid sequence encoding insulin analog or derivatives thereof and further encoding one or more agents for processing the modified pro-insulin to functional insulin analog or derivatives thereof are linked to a promoter capable of controlling expression of the insulin polypeptide in a mammalian host cell.

The promoter may be any DNA sequence, which shows transcriptional activity in the host cell of choice and may be derived from genes encoding proteins either homologous or heterologous to the host cell, and includes mutant, truncated, and hybrid promoters. The promoter may be obtained from genes encoding extra-cellular or intra-cellular polypeptides either homologous or heterologous to the host cell. Accordingly, the present disclosure also provides a chimeric nucleic acid sequence encoding insulin analogs or derivative thereof linked to a promoter capable of controlling expression in a mammalian host cell. Promoters that may be used herein will be generally recognized in the art and include any promoter capable of controlling expression of polypeptides in mammalian cells. Certain genetic elements capable of enhancing expression of the insulin analog polypeptide or derivative thereof may be used herein.

In certain aspects, the chimeric nucleic acid sequences comprising a promoter capable of controlling expression in mammalian host cell linked to a nucleic add sequence encoding an insulin analog polypeptide or derivatives thereof encoding one or more agents for processing the modified pro-insulin to functional insulin analog or derivatives thereof can be integrated into a recombinant expression vector which ensures good expression in a mammalian cell. In some aspects, the chimeric nucleic acid sequences comprising a promoter capable of controlling expression in mammalian host cell linked to a nucleic acid sequence encoding an pro-insulin analog polypeptide or derivatives thereof and further encoding the one or more agents for processing the modified proinsulin to functional insulin analog or derivatives thereof can be integrated into a recombinant expression vector which ensures good expression in a mammalian cell.

In some aspects, the recombinant expression vector according to the disclosure comprises a nucleic acid sequence encoding a cleavable linker connecting the pro-insulin polypeptide and the agents for processing the pro-insulin to functional insulin analog or derivatives thereof. The cleavable linker may be a peptide, a polypeptide or a part of a polypeptide, which is cleaved after the generation of the protein or polypeptide. Particularly, the cleavable linker is a self-cleavable, self-cleaving, self-cleavage peptide or linker, these terms being used interchangeably herein. In one aspect, the cleavable linker comprises a 2A peptide. “2A” or “2A-like” sequences are part of a large family of peptides that can cause peptide bond-skipping. Particularly, the mechanism of 2A-mediated “self-cleavage” was recently discovered to be ribosome skipping the formation of a glycyl-prolyl peptide bond at the C-terminus of the 2A peptide. The 2A-peptide-mediated cleavage commences after the translation. Successful skipping and recommencement of translation results in two “cleaved” proteins: the protein upstream of the 2A is attached to the complete 2A peptide except for the C-terminal proline, and the protein downstream of the 2A is attached to one proline at the N-terminus. Several 2A peptides have been identified in picornaviruses, insect viruses and type C rotaviruses. Examples of cleavable linker according to the disclosure include, but are not limited to, porcine teschovirus-1 2A (P2A), FMDV 2A (F2A); equine rhinitis A virus (ERAV) 2A (E2A); and Thosea asigna virus 2A (T2A), cytoplasmic polyhedrosis virus 2A (BmCPV2A) and flacherie Virus 2A (BmIFV2A), or a combination thereof, for example such as described in Kim et al. (2011) PLoS ONE and in Liu et al (2017) Sci Rep. 2017.

The selection and construction of expression vectors comprising the heterologous nucleic acid sequences disclosed herein and suitable for use in mammalian host cells are known in the art. Expression vectors usually comprise a plasmid origin of DNA replication, a selectable marker (for e.g., antibiotic selection marker or eGFP), and a promoter and transcriptional terminator separated by a multi-cloning site (expression cassette) and a DNA sequence encoding at least one ribosome binding site. Transcription of the heterologous nucleic acid (gene of interest, e.g., nucleic add sequence encoding an insulin analog polypeptide or derivatives thereof) is usually controlled by a regulated promoter, which allows cell growth to be separated from product synthesis resulting in higher yields than if the protein was constitutively expressed.

A recombinant expression system is selected from eukaryotic hosts. In certain aspects, the eukaryotic host includes mammalian cells. Commercial sources of mammalian cells used for recombinant protein expression also provide instructions for usage of the cells. The choice of the expression system depends on the features desired for the expressed polypeptide.

The term “recombinant”, as used herein to describe a protein or polypeptide means a polypeptide produced by expression of a recombinant polynucleotide. The term “recombinant”, as used herein in reference to cells, means cells that can be or have been used as recipients for recombinant vectors or other transfer DNA, and include progeny of the original cell which has been transfected. It shall be understood that progeny of a single parental cell may not be completely identical in morphology or in genomic or total DNA complement to the original parent, due to accidental or deliberate mutation. Progeny of a parental cell which are sufficiently similar to the parent to be characterized by a relevant property, such as the presence of a nucleotide sequence encoding a desired polypeptide, are also considered progeny.

Accordingly, the present disclosure includes recombinant expression vectors comprising in the 5′ to 3′ direction of transcription as operably linked components: (i) a nucleic acid sequence capable of controlling expression in mammalian cells; and (ii) a nucleic acid sequence encoding a pro-insulin polypeptide or derivative thereof; wherein the expression vector is suitable for expression in a mammalian host cell. Accordingly, the present disclosure also encompasses a recombinant expression vector comprising in the 5′ to 3′ direction of transcription as operably linked components: (i) a nucleic acid sequence capable of controlling expression in a mammalian host cell; (ii) a nucleic acid sequence encoding at least one pro-insulin polypeptide or derivative thereof, and (iii) a nucleic acid sequence encoding one or more agents for processing the modified pro-insulin to functional insulin analog or derivatives thereof, wherein the expression vector is suitable for expression in a mammalian host cell.

To direct the insulin analog or derivative thereof into the secretory pathway of the host cells, a secretory signal sequence (also known as a leader sequence, prepro sequence or pre sequence) may be provided in the expression vector. The secretory signal sequence is joined to the DNA sequence encoding the pro-insulin in the correct reading frame. Secretory signal sequences are commonly positioned 5′ to the DNA sequence encoding the peptide. The signal peptide may be naturally occurring signal peptide, or a functional part thereof, or it may be a synthetic peptide. Accordingly, in some aspects, the expression vectors of the present disclosure further comprise a nucleic acid sequence encoding a signal peptide at the N-terminus of the nucleic acid sequence encoding a pro-insulin polypeptide. A signal peptide of the present disclosure allows the cell to secrete the insulin molecule, and is removed from the mature molecule during the secretion process. Table 4 lists some commonly used signal peptide sequences for efficient secretion of a recombinant protein expressed in mammalian cells.

TABLE 4
Exemplary signal peptides

| | | SEQ |
|---|---|---|
| Leader sequence | | ID |
| Name | Sequence | NO |
| Human OSM | MGVLLTQRTLLSLVLAL | 16 |
| | LFPSMASM | |
| VSV-G | MKCLLYLAFLFIGVNC | 17 |
| Mouse Ig Kappa | METDTLLLWVLLLWVPGS | 18 |
| | TGD | |
| Mouse Ig Heavy | MGWSCIILFLVATATGVHS | 19 |
| BM40 | MRAWIFFLLCLAGRALA | 20 |
| Secrecon | MWWRLWWLLLLLLLLWPMV | 21 |
| | WA | |
| Human IgKVIII | MDMRVPAQLLGLLLLWLRG | 22 |
| | ARC | |
| CD33 | MPLLLLLPLLWAGALA | 23 |
| tPA | MDAMKRGLCCVLLLCGAVF | 24 |
| | VSPS | |
| Human | MAFLWLLSCWALLGTTFG | 25 |
| Chymotrypsinogen | | |
| Human | MNLLLILTFVAAAVA | 26 |
| trypsinogen-2 | | |
| Human IL-2 | MYRMQLLSCIALSLALVI | 27 |
| | NS | |
| Gaussia luc | MGVKVLFALICIAVAEA | 28 |
| Albumin(HSA) | MKWVTFISLLFSSAYS | 29 |
| Influenza | MKTIIALSYIFCLVLG | 30 |
| Haemagglutinin | | |
| Human insulin | MALWMRLLPLLALLALWG | 31 |
| | PDPAAA | |
| Silkworm | MKPIFLVLLVVTSAYA | 32 |
| Fibroin LC | | |

In some aspects, the expression vectors of the present disclosure further comprises in the 5′ to 3′ direction of transcription a nucleic acid sequence encoding a N-terminal fusion partner connected to the polypeptide to generate a fusion protein. In certain aspects, the fusion partner is present during translation but is cleaved off by intracellular protease, such as furin, during protein export, thereby releasing the polypeptide of interest. Non-limiting examples of N-terminal fusion partners include human siderocalin (SCN), murine siderocalin, chicken Ex-FABP, or quail Q83. SCN fusion facilitates enhanced protein folding and rapid protein expression. Details of producing SCN fusion proteins are described in U.S. Pat. No. 10,156,559, the contents of which are hereby expressly incorporated by reference in their entirety for any purpose.

Accordingly, in some aspects, the present disclosure provides an expression vector comprising in the 5′ to 3′ direction of transcription as operably linked components: (i) a nucleic acid sequence capable of controlling expression in a mammalian host cell; (ii) a nucleic acid sequence encoding a signal peptide for efficient secretion of insulin analog or derivative thereof; (iii) optionally, a nucleic acid sequence encoding a N-terminal fusion partner; (iv) a nucleic acid sequence encoding a modified pro-insulin polypeptide; and (v) a nucleic acid sequence encoding one or more agents for processing the modified pro-insulin to functional insulin analog or derivatives thereof. In some aspects, the agent for processing the modified pro-insulin to functional insulin analog or derivatives thereof is selected from the group consisting of Prohormone Convertase 1/3 (PC1/3), PC2 and carboxypeptidase E (CPE). In some aspects, the agents for processing the modified pro-insulin to functional insulin lispro or derivatives thereof comprise PC1/3 and CPE and, optionally, PC2. In another aspect, the agents for processing the modified pro-insulin to functional insulin glargine or derivatives thereof comprise PC1/3 and, optionally, PC2.

In some aspects, the present disclosure provides a first and a second expression vectors. In some aspects, the first expression vector comprises in the 5′ to 3′ direction of transcription as operably linked components: (i) a nucleic acid sequence capable of controlling expression in mammalian cells; (ii) a nucleic acid sequence encoding a signal peptide for efficient secretion of insulin analog or derivative thereof, (iii) optionally, a nucleic acid sequence encoding a N-terminal fusion partner; and (iv) a nucleic acid sequence encoding a modified pro-insulin polypeptide. In another aspect, the second expression vector comprises in the 5′ to 3′ direction of transcription as operably linked components: (i) a nucleic acid sequence capable of controlling expression in mammalian cells; and (ii) a nucleic acid sequence encoding one or more agents for processing the modified pro-insulin to functional insulin analog or derivatives thereof. In some aspects, the agent for processing the modified pro-insulin to functional insulin analog or derivatives thereof is selected from a group consisting of PC1/3, PC2 and CPE. In some aspects, the agents for processing the modified pro-insulin to functional insulin lispro or derivatives thereof comprise PC1/3 and CPE and, optionally, PC2. In another aspect, the agents for processing the modified pro-insulin to functional insulin glargine or derivatives thereof comprise PC1/3 and, optionally, PC2.

In some aspects, the present disclosure provides methods for manufacturing insulin analog and/or a derivative thereof comprising producing competent mammalian host cells that express and secret insulin comprising transforming the host cells with an expression vector co-expressing a modified pro-insulin polypeptide and one or more agents for processing the modified pro-insulin to functional insulin analog or derivatives thereof. In another aspects, the present disclosure provides methods for manufacturing insulin analog and/or a derivative thereof comprising producing competent mammalian host cells that express and secret insulin comprising transforming the host cells with a first expression vector expressing a modified pro-insulin polypeptide and a second expression vector expressing one or more agents for processing the modified pro-insulin to functional insulin analog or derivatives thereof.

In some aspects, the present disclosure provides a method for manufacturing rapid-acting insulin analog and/or a derivative thereof comprises transforming mammalian host cells with an expression vector comprising (i) a nucleic acid sequence capable of controlling expression in a mammalian host cell; (ii) a nucleic acid sequence encoding a signal peptide for efficient secretion of insulin analog or derivative thereof; (iii) optionally, a nucleic acid sequence encoding a N-terminal fusion partner; (iv) a nucleic acid sequence encoding a modified pro-insulin polypeptide; (v) a nucleic acid sequence encoding PC1/3; (vi) a nucleic acid sequence encoding CPE; and (vii) optionally, a nucleic acid sequence encoding PC2. In some aspects, the rapid-acting insulin analog is insulin lispro.

In some aspects, the present disclosure provides a method for manufacturing long-acting insulin analog and/or a derivative thereof comprises transforming mammalian host cells with a recombinant expression vector comprising (i) a nucleic acid sequence capable of controlling expression in a mammalian host cell; (ii) a nucleic acid sequence encoding a signal peptide for efficient secretion of insulin analog or derivative thereof; (iii) optionally, a nucleic acid sequence encoding a N-terminal fusion partner; (iv) a nucleic acid sequence encoding a modified pro-insulin polypeptide; (v) a nucleic acid sequence encoding PC1/3; and (vi) optionally, a nucleic acid sequence encoding PC2. In some aspects, the long-acting insulin analog is insulin glargine.

For the production of long-acting insulin, such as insulin glargine that contains two extra arginine molecules at the end of the B chain (Arg B31 and Arg B32), endogenous carboxypeptidase E activity may need to be blocked to maintain the two terminal Arginines. Accordingly, the present disclosure provides a method for manufacturing long-acting insulin analog and/or a derivative thereof further comprises the optional step of blocking endogenous carboxypeptidase E (CPE) activity. In some aspects, the step of blocking endogenous carboxypeptidase E activity comprises introducing a deletion or mutation into endogenous CPE that results in reduced or eliminated expression or activity. In some aspects, the host cell may be a CRISPR-modified host cell having reduced or eliminated CPE activity. Such host cells may be produced, for example, by reducing or eliminating expression of CPE using CRISPR or by permitting expression of a CPE that is catalytically-inactive. For example, the serine at position 202 of the sequence set forth in SEQ ID NO: 9 may be mutated, for example, to Proline. In some aspects, the reduction or elimination of CPE activity may be conditional or inducible (e.g. in the form of Cre dependent constructs). In some aspects, the step of blocking endogenous carboxypeptidase E activity comprises co-expressing a mutated CPE with at least one mutation at a position selected from a group consisting of 72H, 75E, 147R, 192H, 202S, 243Y, and 296E of the sequence set forth in SEQ ID NO: 9 in the mammalian host cells. In some aspects, a nucleic acid sequence encoding the mutated CPE is included in the expression vector co-expressing a modified pro-insulin polypeptide. In another aspect, a nucleic acid sequence encoding the mutated CPE is included in a separate expression vector. In some aspects, the present disclosure provides a host cell expressing a catalytically-inactive CPE, wherein the CPE binds substrate but does not cleave a peptide bond, wherein the catalytically-inactive CPE has at least one mutation at a position selected from a group consisting of 72H, and 75E, 147R, 192H, 202S, 243Y, and 296E of the sequence set forth in SEQ ID NO: 9. In some aspects, the step of blocking endogenous CPE activity comprises adding a blocking agent selected from a group consisting of dopamine quinine, dopamine, norepinephrine, epinephrine, potato carboxypeptidase inhibitor (PCI), 9-mer peptide-designated CPI-2KR, and a peptide encoding a decoy arginine sequence. The decoy arginine peptide of the present disclosure comprises a polymer of at 3 amino acids in length and having an amino acid sequence with at least 70% amino acid sequence identity to the C-terminal extension of the B chain of a long-acting insulin, for example, the sequence set forth in SEQ ID NO: 15. The decoy arginine peptide is configured to mimic the C-terminal extension of the B-chain of a long-acting insulin to bind endogenous CPE so that it block the endogenous CPE from removing the C-terminal extension of the B-chain.

In some aspects, the present disclosure provides a mutated CPE or a polynucleotide encoding a mutated CPE, wherein the mutated CPE is catalytically-inactive. In some aspects, the mutated CPE binds to but does not hydrolyze a protein. In some aspects, the mutated CPE comprises at least one mutation at a position selected from a group consisting of 72H, 75E, 147R, 192H, 202S, 243Y, and 296E of the sequence of SEQ ID NO: 9.

The term “suitable for expression in a mammalian host cell” means that the recombinant expression vector comprises the chimeric nucleic acid sequence of the present disclosure linked to genetic elements required to achieve expression in a mammalian cell. Expression of recombinant proteins is often difficult outside their original host. For example, variation in codon usage bias has been observed across different species of bacteria (Sharp et al., 2005, Nucl. Acids. Res. 33:1141-1153). Over-expression of recombinant proteins even within their native host may also be difficult. In certain aspects of the invention, nucleic acids that are to be introduced into host cells may be codon optimized to enhance protein expression. Codon optimization refers to alteration of codons in genes or coding regions of nucleic acids for transformation of an organism to reflect the typical codon usage of the host organism without altering the polypeptide for which the DNA encodes.

Genetic elements that may be included in the expression vector in this regard include a transcriptional termination region, polyadenylation signals, and translational enhancer sequences, one or more nucleic acid sequences encoding marker genes, one or more origins of replication and the like. In certain aspects, the recombinant expression vector may further comprise a DNA sequence enabling the vector to replicate in the mammalian host cell. In some aspects, the expression vector further comprises genetic elements required for the integration of the vector or a portion thereof in the mammalian host cell's nuclear genome.

The recombinant vector may be an autonomously replicating vector, i.e., a vector which exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome. In certain aspects, the recombinant expression vector is capable of replicating in a mammalian host cell. Recombinant vectors suitable for the introduction of nucleic acid sequences into host cells include mammalian expression systems, for e.g., Lentivirus based vectors, pHEK293 Ultra Expression Vectors, and pEF1α-IRES

Host cells are genetically engineered (including but not limited to, transformed, transduced or transfected) with the polynucleotides of the present disclosure or constructs which include a polynucleotide of the present disclosure, including but not limited to, a vector of the present disclosure, which can be, for example, a cloning vector or an expression vector. For example, the coding regions for the orthogonal tRNA, the orthogonal tRNA synthetase, and the protein to be derivatized are operably linked to gene expression control elements that are functional in the desired host cell. The vector can be, for example, in the form of a plasmid, a cosmid, a phage, a bacterium, a virus, a naked polynucleotide, or a conjugated polynucleotide. The vectors are introduced into cells and/or microorganisms by standard methods including electroporation (Fromm et al., Proc. Natl. Acad. Sci. USA 82, 5824 (1985)), infection by viral vectors, high velocity ballistic penetration by small particles with the nucleic acid either within the matrix of small beads or particles, or on the surface (Klein et al., Nature 327, 70-73 (1987)), and/or the like. Techniques suitable for the transfer of nucleic acid into cells in vitro include the use of liposomes, microinjection, cell fusion, DEAE-dextran, the calcium phosphate precipitation method, etc. In vivo gene transfer techniques include, but are not limited to, transfection with viral (typically retroviral) vectors and viral coat protein-liposome mediated transfection [Dzau et al., Trends in Biotechnology 11:205-210 (1993)]. In some situations, it may be desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life.

In certain aspects, the host cell provided in the methods disclosed herein is a mammalian host cell. The mammalian host cell into which the nucleic acid sequence and/or the recombinant expression vector is introduced may be any mammalian cell which is capable of expressing the insulin precursor and/or insulin analogs, and includes, but is not limited to HEK293, CHO, COS and HeLa cells. The present disclosure also provide host cells selected from algae and yeast. The host cells provided in the methods disclosed herein may comprise have genetic modifications to permit the formation of stable disulfide bonds within the cytoplasm.

The recombinant expression vectors, nucleic acid sequences and chimeric nucleic and sequences of the present disclosure may be prepared in accordance with methodologies well known to those skilled in the art of molecular biology. The procedures used to ligate the DNA sequences coding for the insulin product, optionally the sequences encoding the one or more agents for processing the modified pro-insulin to functional insulin analog or derivatives thereof, the promoter, and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989). Such preparation will typically involve but are not limited to the bacterial species Escherichia coli as an intermediary cloning host. The preparation of the E. coli vectors as well as the mammalian transformation vectors may be accomplished using commonly known techniques such as restriction digestion, ligation, gel electrophoresis, DNA sequencing, the Polymerase Chain Reaction (PCR) and other methodologies. These methodologies permit the linking of nucleic acid sequences and polypeptides to which the present disclosure pertains. A wide variety of cloning vectors are available to perform the necessary steps required to prepare a recombinant expression vector. In certain aspects, the methods include in-fusion cloning. In-Fusion Cloning is a highly efficient, ligation-independent cloning method, based on the annealing of complementary ends of a cloning insert and linearized cloning vector. This method ensures easy, single-step directional cloning of any gene of interest into any vector at any locus. In-Fusion constructs are seamless, enabling translational reading frame continuity without any interfering “scar” sequences.

Typically, these cloning vectors contain a marker allowing selection of transformed cells. Nucleic acid sequences may be introduced in these vectors, and the vectors may be introduced in E. coli grown in an appropriate medium. Recombinant expression vectors may readily be recovered from cells upon harvesting and lysing of the cells. Further, general guidance with respect to the preparation of recombinant vectors may be found in, for example: Sambrook et al., Molecular Cloning, a Laboratory Manual, Cold Spring Harbor Laboratory Press, 1989, Vol. 3.

Marker genes that may be used in accordance with the present disclosure include all genes that allow the distinction of transformed cells from non-transformed cells, including all selectable and screenable marker genes. A marker gene may be a resistance marker, such as an antibiotic resistance marker against, for example, kanamycin, ampicillin, G418, bleomycin, hygromycin which allows selection of a trait by chemical means or a tolerance or resistance marker against a chemical agent. Resistance markers, when linked in dose proximity to nucleic acid sequence encoding the insulin polypeptide, may be used to maintain selection pressure on a population of plant cells or plants that have not lost the nucleic acid sequence encoding the insulin analog polypeptide or derivative thereof. Screenable markers that may be employed to identify transformants through visual inspection include 3-glucuronidase (GUS) and green fluorescent protein (GFP and eGFP).

It will be understood that the vector may be constructed either by first preparing a DNA construct containing the entire DNA sequence encoding the insulin precursors of the disclosure, optionally the one or more agents for processing the modified pro-insulin to functional insulin analog or derivatives thereof, and subsequently inserting this fragment into a suitable expression vector, or by sequentially inserting DNA fragments containing genetic information for the individual elements (such as the signal, pro-peptide, modified C-peptide, A and B chains, optionally, the agents for processing the pro-insulin to functional insulin analog or derivatives thereof) followed by ligation.

After a host organism has been chosen, the vector is transferred into the host organism using methods generally known by those of ordinary skill in the art. Examples of such methods may be found in Advanced Bacterial Genetics by R. W. Davis et. al., Cold Spring Harbor Press, Cold Spring Harbor, N.Y., (1980), which is specifically incorporated herein by reference. It is preferred, in one aspect, that the transformation occur at low temperatures, temperature regulation is contemplated as a means of regulating gene expression through the use of operational elements as set forth.

The engineered host cells may be cultured in conventional nutrient media modified as appropriate for such activities as, for example, screening steps, activating promoters or selecting transformants. These cells can optionally be cultured into transgenic organisms. Other useful references, including but not limited to for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla. Mammalian host cells are cultured under conditions appropriate for the expression of the recombinant insulin analog and/or derivatives thereof. These conditions are generally specific for the host organism, and are readily determined by one of ordinary skill in the art, in light of the published literature regarding the growth conditions for such organisms, for example Bergey's Manual of Determinative Bacteriology, 8th Ed., Williams & Wilkins Company, Baltimore, Md., which is specifically incorporated herein by reference.

Examples of suitable mammalian host cells are known to those of ordinary skill in the art. Such host cells may be Chinese hamster ovary (CHO) cells, (e.g. CHO-K1; ATCC CCL-61), Green Monkey cells (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)). These cell lines and others are available from public depositories such as the American Type Culture Collection, Rockville, Md. In order to provide improved glycosylation of the insulin polypeptide, a mammalian host cell may be modified to express sialyltransferase, e.g. 2,6-sialyltransferase, e.g. as described in U.S. Pat. No. 5,047,335, which is incorporated by reference herein.

In certain aspect, the present disclosure provides recombinant mammalian cells, comprising a polynucleotide sequence encoding the desired insulin analog and or derivative thereof. In some aspects, the polynucleotide sequence further encodes at least one agent for processing the modified pro-insulin to functional insulin. An expression vector comprising such polynucleotide sequence is introduced into the host cell so that the vector is maintained as a chromosomal integrant or as a self-replicating extra-chromosomal vector.

In certain aspects, the method further comprises culturing mammalian host cells transformed with the recombinant vectors of the current disclosure under conditions suitable for expression of the transfected recombinant vectors. The expressed recombinant product is then isolated and purified or recovered from the culture broth. In certain aspects, the methods disclosed herein further comprise purifying the recovered expressed product from the culture broth.

In some aspects, the step of purifying the recovered expressed product comprises subjecting the recovered expressed product to a reverse phase HPLC column and eluting a sample that includes the insulin analog and/or derivative thereof with an organic solvent, under conditions that allow the compound to be bound to the resin, and washing the organic solvent from the resin with an aqueous buffer solution. The effective performance of the present invention requires the individuation of right combination of the chromatographic matrix to be used, the pH value and the ionic strength of the buffer for efficient purifications

The production of high amounts of mature insulin or insulin analog in a mammalian cell will significantly reduce the number of downstream purification steps necessary to produce an insulin or insulin analog product of a purity sufficiently high for pharmaceutical purposes. U.S. Pat. No. 4,916,212 discloses a method of making insulin in yeast cells where an insulin precursor is converted into human insulin in two steps i.e. a transpeptidation to convert the single chain insulin precursor B(1-29)-Ala-Ala-Lys-A(1-21) into an ester of human insulin and then a hydrolysis of the insulin ester into human insulin. Each conversion step will require an initial separation step and at least one subsequent purification step. Thus at least six additional steps are necessary to produce the mature insulin including at least one enzymatic conversion.

No enzymatic cleavage runs to a 100% cleavage leaving impurities of uncleaved or partially cleaved impurities which have to be efficiently removed in the case of pharmaceutical products. Thus, each cleavage step will be followed by at least one isolation or purification step, typically a chromatographic purification by means of exchange chromatography, gel filtration chromatography, affinity chromatography, or the like.

Methods for small-scale or large-scale fermentation can also be used in protein expression, including but not limited to, fermentors, shake flasks, fluidized bed bioreactors, hollow fiber bioreactors, roller bottle culture systems, and stirred tank bioreactor systems. Each of these methods can be performed in a batch, fed-batch, or continuous mode process.

Human insulin polypeptides of the invention can generally be recovered using methods standard in the art. For example, culture medium can be centrifuged and/or filtered to remove cellular debris. The supernatant may be concentrated or diluted to a desired volume or diafiltered into a suitable buffer to condition the preparation for further purification. Further purification of the insulin polypeptide of the present invention includes separating deamidated and clipped forms of the insulin polypeptide variant from the intact form.

Any of the following exemplary procedures can be employed for purification of insulin polypeptides of the invention: affinity chromatography; anion- or cation-exchange chromatography (using, including but not limited to, DEAE SEPHAROSE); chromatography on silica; high performance liquid chromatography (HPLC); reverse phase HPLC; gel filtration (using, including but not limited to, SEPHADEX G-75); hydrophobic interaction chromatography; size-exclusion chromatography; metal-chelate chromatography; ultrafiltration/diafiltration; ethanol precipitation; ammonium sulfate precipitation; chromatofocusing; displacement chromatography; electrophoretic procedures (including but not limited to preparative iso electric focusing), differential solubility (including but not limited to ammonium sulfate precipitation), SDS-PAGE, or extraction.

Chromatographic column material for use in commercial scale is very expensive and therefore reduction of the number of such chromatographic steps has a significant impact on the production economy. A reduction of the downstream conversion and purification step will in addition reduced the amount of labor work and hours spent in the process and thus further improve the production economy. Advantageously, the methods and compositions of the present disclosure provide the ability to produce, harvest, and purify mature functional insulin or an analog thereof in high yields directly from the culture broth, thereby requiring fewer downstream process steps necessary to produce a product of sufficient purity for pharmaceutical use.

In some aspects, the present disclosure pertains to a method for obtaining a purified biologically active heterologous recombinant protein expressed in a mammalian expression system. In certain aspects the method comprises culturing the host cells transformed by at least one vector comprising a heterologous nucleic acid sequence encoding a peptide that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 1, SEQ ID: No. 2; SEQ ID: No. 3, and SEQ ID: No. 4. In certain aspects, the method comprises culturing the host cells transformed by at least one vector comprising the nucleotide sequence encoding a peptide that has an amino acid sequence as set forth in SEQ ID NO: 1, SEQ ID NO: 2, SEQ ID NO: 3, and SEQ ID: No. 4. In certain aspects, the vector comprising the heterologous nucleic acid encoding the peptide further encodes agents for processing the heterologous protein to a mature functional protein.

In certain aspect, the method further comprises recovering the expressed heterologous protein, wherein the step of recovering the protein comprises separating the protein from the host cell to produce a recovered protein preparation. In some aspects, the method further comprises purifying the recovered protein preparation to remove at least one related impurity by contacting the recovered protein preparation with a chromatographic matrix. Purification is carried out by applying a polar organic buffer solvent in the aqueous phase containing the organic acid buffer and precipitating the eluted protein. In any of the presently disclosed aspects, the mature functional protein is human insulin analog and/or derivative thereof.

According to one aspect of the current disclosure, the yield of the purified insulin analog and/or derivative thereof is 75%-100%. According to another aspect of the present disclosure, the yield of the purified insulin analog and/or derivative thereof is 75%-80%, 80%-85%, 85%-90%, 90%-95%, or 95%-100%.

In certain aspects, the present disclosure provides an integrated and continuous process for manufacturing a recombinant human insulin analog and/or derivative thereof, the process comprising: (i) culturing recombinant human insulin analog-secreting mammalian cells in a perfusion bioreactor comprising a liquid culture medium under conditions that allow the cells to secrete the recombinant the insulin analog and/or derivative thereof into the medium, and wherein substantially cell-free volumes of the medium are continuously or periodically removed from the perfusion bioreactor and fed into a first periodic counter current chromatography system (PCCS1); (ii) capturing the recombinant insulin analog and/or derivative thereof from the medium using the PCCS1, wherein an eluate of the PCCS1 comprising the recombinant insulin analog and/or derivative thereof is continuously fed into a second periodic counter current chromatography system (PCCS2); and (iii) purifying the recombinant insulin analog and/or derivative thereof using the PCCS2, wherein the purifying is performed using a resin in the PCCS2 that is different in chemical structure compared to the resin in the PCCS2 used to perform the polishing, and an eluate from the PCCS2 is the insulin analog and/or derivative thereof; and wherein the process is integrated and runs continuously from the culturing step to the eluate from the PCCS2 that is the insulin analog and/or derivative thereof. In certain aspects, the recombinant human insulin analog-secreting mammalian cells are transformed by at least one vector comprising a heterologous nucleic acid sequence encoding a peptide that has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 1, SEQ ID: No. 2; SEQ ID: No. 3, and SEQ ID: No. 4. In certain aspects, the recombinant human insulin analog-secreting mammalian cells are transformed by at least one vector comprising a heterologous nucleic acid sequence encoding a peptide comprising the amino acid sequence as set forth in SEQ ID NO: 1, SEQ ID NO: 2, SEQ ID NO: 3, and SEQ ID: No. 4. In certain aspect, the at least one vector comprising the heterologous nucleic acid sequence, further encodes at least one agent for processing the modified pro-insulin to functional insulin. In an alternative aspect, the recombinant human insulin analog-secreting mammalian cells are further transformed by a second vector comprising a heterologous nucleic acid sequence encoding at least one agent for processing the modified pro-insulin to functional insulin. In some aspects, the at least one agent encoded by the heterologous nucleic acid has at least about 85% (i.e., at least about 85%, 86%, 87%, 89%, 90%, 91%, 92%, 93%, 94%, 95%, 96%, 97%, 98%, 99%, or more) identity to the amino acid sequence set forth in SEQ ID: No. 5, SEQ ID: No. 6; SEQ ID: No. 7, and SEQ ID: No. 8. In certain aspects, the at least one agent encoded by the heterologous nucleic acid has an amino acid sequence as set forth in SEQ ID: No. 5, SEQ ID: No. 6; SEQ ID: No. 7, and SEQ ID: No. 8.

Pharmaceutical insulin formulations may be prepared from the purified insulin and/or purified insulin analog and such formulations may be used to treat diabetes. Generally the purified insulin will be admixed with a pharmaceutically acceptable carrier or diluent in amounts sufficient to exert a therapeutically useful effect in the absence of undesirable side effects on the patient treated. To formulate an insulin composition, the weight fraction of insulin is dissolved, suspended, dispersed or otherwise mixed in a selected carrier or diluent at an effective concentration such that the treated condition is ameliorated. The pharmaceutical insulin formulations are preferably formulated for single dosage administration. Therapeutically effective doses for the parenteral delivery of human insulin are well known to the art. Where insulin analogs are used or other modes of delivery are used therapeutically effective doses may be readily empirically determined by those of skill in the art using known testing protocols or by extrapolation of in vivo or in-vitro test data. It is understood however that concentrations and dosages may vary in accordance with the severity of the condition alleviated. It is further understood that for any particular subject, specific dosage regimens may be adjusted over time according to individual judgement of the person administering or supervising administration of the formulations.

Pharmaceutical solutions or suspensions may include for example a sterile diluent such as, for example, water, lactose, sucrose, dicalcium phosphate, or carboxymethyl cellulose. Carriers that may be used include water, saline solution, aqueous dextrose, glycerol, glycols, ethanol and the like, to thereby form a solution or suspension. If desired the pharmaceutical compositions may also contain non-toxic auxiliary substances such a wetting agents; emulsifying agents; solubilizing agents; antimicrobial agents, such as benzyl alcohol and methyl parabens; antioxidants, such as ascorbic acid and sodium bisulfite; chelating agents such as ethylenediaminetetraacetic acid (EDTA), zinc ions; pH buffering agents such as sodium hydroxide, hydrochloric acid, acetate, citrate or phosphate buffers; and combinations thereof.

The final formulation of the insulin preparation will generally depend on the mode of insulin delivery. The insulin prepared in accordance with the present disclosure may be delivered in any desired manner; however parenteral, form of delivery are considered the most likely used modes of delivery. The insulin analogs produced by the method according to the present disclosure may be used in the treatment of states that are sensitive to insulin. Thus, they can be used in the treatment of type 1 diabetes, type 2 diabetes and hyperglycemia for example as sometimes seen in seriously injured persons and persons who have undergone major surgery. Where expedient, the insulin analogs may be used in mixture with other types of insulin, e.g. insulin analogs with a more rapid onset of action. Examples of such insulin analogs are described e.g. in the European patent applications having the publication Nos. EP 214826, EP 375437 and EP 383472.

Pharmaceutical compositions containing the insulin analogs of this disclosure can be used in the treatment of states which are sensitive to insulin. Thus, they can be used in the treatment of type 1 diabetes, type 2 diabetes and hyperglycaemia for example as sometimes seen in seriously injured persons and persons who have undergone major Surgery. The optimal dose level for any patient will depend on a variety of factors including the efficacy of the specific insulin analog employed, the age, body weight, physical activity, and diet of the patient, on a possible combination with other drugs, and on the severity of the state to be treated. It is recommended that the daily dosage of the insulin derivative may be determined for each individual patient by those skilled in the art in a similar way as for known insulin compositions.

### EXAMPLE

### Example 1: HEK293 Cell Transfection or Transduction

Procedures to introduce a recombinant expression vector into mammalian host cells (e.g. HEK293) are well known in the art. The transformation of the host cells can be carried out by numerous procedures known for those skilled in the art including non-viral methods or via a viral vector.

For non-viral transfection, 10,000 to 15,000 HEK293 cells were plated per well of a 24-well plate, in 0.5 ml of complete growth medium 12-24 hours prior to transfection. The cells were washed with PBS 1× and 0.5 ml of fresh growth medium added to each well. Transfection complexes were prepared by mixing 40 μl of serum-free medium, 4.5 μl of transfection reagent, and (referred to a final volume including growth medium), 500 ng expression vector. Transfection complexes were incubated at room temperature (RT) for 15-30 minutes. 2 μl of a complex condenser reagent was added. The complex condenser reagent increases transfection efficiency by reducing the size of transfection complex; however, it may increase cell toxicity. Following incubation, prepared transfection complexes were added to the 0.5 ml of complete growth medium per well of the washed HEK293 cells. The cells were incubated with the transfection complexes at 37° C. in a humidified CO₂ incubator. Successfully transfected cells were selected using an appropriate marker, for example through cell sorting in case a fluorescent marker is used or using an antibiotic resistance marker or culturing. A fraction of the successfully transfected cells were assayed for target gene expression 48-72 hours post transfection.

For viral transduction, 4.5×10⁶ cells 293T cells were split one day before transfection into 10 cm dish (9 ml). On the day of transfection, total plasmid DNA (ug) in 500 ul was diluted. The transfer vectors include viral packaging (psPAX2):viral envelope (pMD2G) at 4:2:1 ratio (6:3:1.5 ug, respectively). 42 ul of PEI (1 ug/uL) was added to the diluted DNA and was mixed immediately by pipetting up and down/vortexing. The volume of PEI used is based on a 4:1 ratio of PEI(ug):total DNA(ug). The DNA/PEI mixture was incubated for 10-15 minutes at RT and 500 ul of DNA/PEI mixture was added to each plate of cells for incubation overnight. The media was aspirated and the cells were washed with pre-warmed 8 ml PBS once. Then the cells were overlaid with pre-warmed 9-10 ml transfection media (DMEM w/o Phenol Red supplemented with 3 to 4% FBS and glutamine) and incubated for 18-24 hours. Then viral supernatant was collected and filtered using 0.22 μm, and stored at 4° C. Optionally, viral supernatant was aliquoted into 50 ml conical tubes and spun overnight at 4° C. at 8500 g, media was aspirated carefully (pellet can be loose), and resuspend at 100× in HBSS. Virus can be aliquoted and stored at −80 indefinitely. HEK293 cells were plated and infected at 50-80% confluence. Virus was thawed in water bath. Polybrene was added to virus supernatant at 6 ug/ml final concentration and viral supernatant/polybrene mix was filtered in 0.45 uM syringe filter. The cells were washed with 2 ml fresh DMEM complete media and 2 ml viral supernatant/polybrene mix was added. The cells was spun at 1800 rpms (750×g) for 1 hour at RT and incubated at 37 degrees overnight. Then all media was aspirated from wells and the cells were washed with 1×PBS. 2 ml fresh DMEM complete media was added to the cells. The cells were incubated for another 24 hours before being trypsinized and analyzed on FACS.

### Example 2—Production of Human Analog Insulin 50/500 ml on Shaker

HEK293 cells successfully transfected with the insulin analog construct were cultured using appropriate growth media and culture condition and expanded to 0.5×10⁶ cells/mL in 500 ml media. Fresh media was added every 3-4 days as necessary. The expressed insulin analogs secreted into the growth media were harvested after 1-4 weeks. Briefly, the media was collected and filtered through a 0.22 um filter. SDS Page gel to check for sufficient protein production

### Example 3—Purification

The insulin analogs were precipitated from the growth media in the following manner: zinc chloride solution (18%) was added to glargine insulin to a final concentration of 0.1%. The glargine containing media was adjusted to a pH 6.1, while the lispro containing media was adjusted to a pH of 12.1 and incubated at 4° C. for 16 h and then centrifuged at 20,000×g for 30 min. The supernatant was removed and the precipitate containing insulin was collected. The pellet containing glargine was resolved with sample buffer (7 M urea, 0.25 M acetic acid, pH 2.5) and lispro (7 M urea, 0.25 M acetic acid, pH 5.5) for ion-exchange chromatography.

Insulin was purified from the sample buffer using a cation column packed with Sp Sepharose Fast Flow resin (GE Healthcare Bio-Sciences, USA) into XK 16 columns (GE Healthcare Bio-Sciences, USA) on an AKTA avant (GE Healthcare Bio-Sciences). The insulin containing buffer solution was loaded at a flow rate of 1 ml/min on a 50 ml SP Sepharose column equilibrated at a flow rate of 5 ml/min with 10 CV (column volume) equilibration buffer (7 M urea and 0.25 M acetic acid, pH 2.5/5.5). The column was washed at a flow rate of 5 ml/min with 10 CV elution buffer A (7 M urea and 0.25 M acetic acid, pH 2.5/5.5), and then bound insulin was eluted at a flow rate of 5 ml/min with 6 CV elution buffers A and B (7 M urea, 0.25 M acetic acid, and 1 M sodium chloride, pH 2.5/5.5) by application of a linear gradient (0-1M NaCl). The eluent was monitored at 280 nm and each peak was collected in fraction tubes. The collected fractions were analyzed by HPLC with a Protein & Peptide C4 analytical column.

### Example 4—Preparative High-Performance Liquid Chromatography (Prep-HPLC)

The fractions collected by cation-exchange chromatography that contained more than 60% purity of insulin were pooled based on the purity of HPLC analysis. Prep-HPLC was carried out on an Agilent 1200 system (USA) equipped with a C8 prep HT column (21.2 mm×150 mm, particle size 5 m) (Agilent Technologies, USA). Solvent A was prepared with 0.25 M acetic acid and 15% acetonitrile (ACN) and solvent B was prepared with 0.25 M acetic acid and 45% ACN. The column was equilibrated at a flow rate of 3 ml/min with 10 CV solvent A and the collected protein was loaded onto the column at a flow rate of 1 ml/min. After washing at a flow rate of 3 ml/min with 10 CV solvent A, bound proteins were eluted at a flow rate of 3 ml/min with 6 CV solvent A and B by application of a linear gradient (0%-70% solution B). The eluent was monitored at 280 nm and each peak was collected.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of producing a functional, recombinant protein in its native conformation comprising an insulin analog and/or a derivative thereof in competent host cells using a modified human pro-insulin polypeptide comprising:
(a) transforming the competent host cells with an expression vector, wherein the expression vector is suitable for use in the host cells and comprises a heterologous nucleic acid having a sequence encoding the modified human pro-insulin polypeptide;
(b) growing the transformed host cells to express and secret the insulin analog and/or derivative thereof into growth media; and
(c) harvesting the secreted insulin analog and/or derivative thereof from the growth media, wherein the secreted insulin analog and/or derivative thereof is a functional insulin analog and/or derivative thereof in its native conformation and is capable of binding to an insulin receptor peptide.

2. The method of claim 1, wherein the expression vector comprising the heterologous nucleic acid encoding the modified human pro-insulin polypeptide further comprises a heterologous nucleic acid sequence encoding one or more agents for processing the modified proinsulin polypeptide to functional insulin.

3. The method of claim 2, wherein the one or more agents for processing the modified pro-insulin polypeptide to functional insulin are selected from an endoprotease and a carboxypeptidase.

4. The method of claim 1, further comprising co-transforming the competent host cells with a second expression vector comprising a heterologous nucleic acid sequence encoding one or more agents for processing the modified proinsulin to functional insulin.

5. The method of claim 4, wherein the one or more agents for processing the modified pro-insulin to functional insulin are selected from an endoprotease and a carboxypeptidase.

6. The method of claim 3 or 5, wherein the endoprotease is selected from a group consisting of Prohormone Convertase 1/3 (PC1/3) and Prohormone Convertase 2 (PC2).

7. The method of claim 3 or 5, wherein the heterologous nucleic acid sequence encoding one or more agents for processing the modified pro-insulin to functional insulin encodes the sequence of SEQ ID NO: 5.

8. The method of claim 3 or 5, wherein the heterologous nucleic acid sequence encoding one or more agents for processing the modified pro-insulin to functional insulin encodes the sequence of SEQ ID NO: 6.

9. The method of claim 3 or 5, wherein the carboxypeptidase is carboxypeptidase E (CPE).

10. The method of claim 3 or 5, wherein the heterologous nucleic acid sequence encoding one or more agents for processing the modified pro-insulin to functional insulin encodes the sequence of SEQ ID NO: 7 or 8.

11. The method of claim 2 or 4, wherein the heterologous nucleotide sequence encoding the modified human pro-insulin polypeptide encodes the sequence of SEQ ID: NO. 1; and wherein one or more agents for processing the modified human pro-insulin polypeptide to functional insulin comprise PC1/3 and CPE and, optionally, PC2.

12. The method of claim 11, wherein the heterologous nucleic acid sequence encoding one or more agents for processing the modified human pro-insulin to functional insulin encodes the sequences of SEQ ID NO: 5 and SEQ ID NO: 7 or 8 and, optionally, SEQ ID NO: 6.

13. The method of claim 2 or 4, wherein the heterologous nucleotide sequence encoding the modified human proinsulin polypeptide encodes the sequence of SEQ ID: NO. 3; and wherein one or more agents for processing the modified human pro-insulin polypeptide to functional insulin comprise PC1/3 and, optionally, PC2.

14. The method of claim 13, wherein the heterologous nucleic acid sequence encoding one or more agents for processing the modified human pro-insulin to functional insulin encodes the sequences of SEQ ID NO: 5 and, optionally, SEQ ID NO: 6.

15. The method of claim 13, further comprising the step of blocking endogenous carboxypeptidase E activity in the competent host cells.

16. The method of claim 15, wherein the blocking step comprises introducing a deletion or mutation into endogenous CPE at position 202 of the sequence of SEQ ID NO: 9 via CRISPR-based homologous recombination into the competent host cells.

17. The method of claim 16, wherein the mutation at position 202 of the sequence of SEQ ID NO: 9 is S202P mutation.

18. The method of claim 15, wherein the blocking step comprises co-expressing a mutated CPE with at least one mutation at a position selected from a group consisting of 72H, 75E, 147R, 192H, 202S, 243Y, and 296E of the sequence of SEQ ID NO: 9 in the competent host cells.

19. The method of claim 15, wherein the blocking step comprises adding a blocking agent selected from a group consisting of dopamine quinine, dopamine, norepinephrine, epinephrine, potato carboxypeptidase inhibitor (PCI), 9-mer peptide-designated CPI-2KR, and a peptide encoding a decoy arginine sequence.

20. The method of any one of claims 1-19, wherein the expression vector is selected from a lentiviral vector system.

21. The method of any one of claims 1-20, wherein the competent host cells are mammalian cells selected from a group consisting of HEK293 cells, CHO cells, COS, and HeLa cells.

22. The method of any one of claims 1-14 and 20, wherein the competent host cells are selected from a group consisting of algae and yeast.

23. The method of any one of claims 1-22, wherein the competent host cell comprises a genetic modification to facilitate the formation of stable disulfide bonds within the cytoplasm for processing the modified pro-insulin to functional insulin.

24. The method of any one of claims 1-23, wherein the step of harvesting the insulin analog and/or derivative thereof comprises separating the insulin from the transformed mammalian cells.

25. The method of any one of claims 1-24, wherein the insulin analog and/or a derivative thereof is produced continuously.

26. The method of any one of claims 1-25, wherein the insulin analog and/or derivative thereof is effective in treating diabetes, prediabetes, and hyperglycemia

27. A polynucleotide encoding
(i) a modified human pro-insulin polypeptide encoded by the sequence of SEQ ID:1 or SEQ ID:3; and
(ii) at least one agent for processing the modified human pro-insulin to functional insulin.

28. The polynucleotide of claim 27, wherein the at least one agent for processing the modified human pro-insulin to functional insulin is selected from a group consisting of an endoprotease and a carboxypeptidase.

29. The polynucleotide of claim 28, wherein the endoprotease is selected from a group consisting of PC1/3 and PC2.

30. The polynucleotide of claim 29, wherein the PC1/3 is encoded by the sequence of SEQ ID NO: 5.

31. The polynucleotide of claim 29, wherein the endoprotease is PC2.

32. The polynucleotide of claim 31, wherein the PC2 is encoded by the sequence of SEQ ID NO: 6.

33. The polynucleotide of claim 28, wherein the carboxypeptidase is CPE.

34. The polynucleotide of claim 33, wherein the CPE is encoded by the sequence of SEQ ID NO: 7 or 8.

35. The polynucleotide of claim 27, wherein the modified human pro-insulin polypeptide comprises the sequence of SEQ ID: NO. 1; and wherein the at least one agent for processing the modified human pro-insulin to functional insulin comprises PC1/3 and CPE and, optionally, PC2.

36. The polynucleotide of claim 35, wherein the at least one agent for processing the modified human pro-insulin to functional insulin is encoded by the sequences of SEQ ID NO: 5 and SEQ ID NO: 7 or 8 and, optionally, SEQ ID NO: 6.

37. The polynucleotide of claim 27, wherein the modified human pro-insulin peptide comprises the sequence of SEQ ID: NO. 3; and wherein the at least one agent for processing the modified human pro-insulin to functional insulin comprises PC1/3 and, optionally, PC2.

38. The polynucleotide of claim 37, wherein the at least one agent for processing the modified pro-insulin to functional insulin is encoded by the sequences of SEQ ID NO: 5 and, optionally, SEQ ID NO: 6.

39. The polynucleotide of claim 37, further encoding a blocking agent configured to block endogenous carboxypeptidase E activity.

40. The polynucleotide of claim 39, wherein the blocking agent comprises a mutated CPE with at least one mutation at a position selected from a group consisting of 72H, 75E, 147R, 192H, 202S, 243Y, and 296E of the sequence of SEQ ID NO: 9.

41. An expression vector comprising the polynucleotide of any one of claim 27-40.

42. The expression vector of claim 41, wherein the vector is selected from a lentiviral vector system,

43. A modified host cell comprising the polynucleotide of any one of claims 27-40 or the expression vector of claim 41 or 42.

44. The modified host cell of claim 43, wherein the host cell is a mammalian cell.

45. The modified host cell of claim 43 or 44, comprising a genetic modification to facilitate formation of stable disulfide bonds within the cytoplasm for processing the modified proinsulin to functional insulin.

46. The modified host cell of any one of claims 43-45, wherein the modified host cell is selected from HEK293 cells, CHO cells, COS, and HeLa cells.

47. The modified host cell of claim 43, wherein the modified host cell is selected from algae and yeast.

48. The modified host cell of claim 44, further comprising a deletion or mutation into endogenous CPE at position 202 of the sequence set forth in SEQ ID NO: 9 via CRISPR-based homologous recombination.

49. The modified host cell of claim 44, wherein expression and/or activity of the endogenous CPE is reduced or eliminated.

50. The modified host cell of claim 44, wherein the mutated CPE is catalytically-inactive.

51. The modified host cell of claim 44, wherein the mutated CPE binds to but does not hydrolyze a C-terminal extension of a B-chain of a long-acting insulin.

52. A method of treating diabetes in a subject in need thereof, said method comprising administering to said subject a therapeutically effective amount of the human insulin analog obtained by the method of claim 1.

53. A method of restoring glucose homeostasis in a subject in need thereof, comprising administering a therapeutically effective amount of the human insulin analog obtained by the method of claim 1.

54. A method of producing a protein in its native conformation in host cells using a propeptide comprising:
(a) growing the host cells recombinantly expressing the propeptide and a protease under conditions sufficient to allow maturation and secretion of the protein; and
(b) harvesting the secreted protein from growth media.

55. The method of claim 54, wherein the protein is selected from a group consisting of insulin, amylin, gastrin, ghrelin, glucagon, somatostatin, α-MSH, ACTH, and β-endorphin

56. The method of claim 54, wherein the protease comprises an endoprotease and a carboxypeptidase.

57. The method of claim 56, wherein the endoprotease is selected from a group consisting of PC1/3 and PC2.

58. The method of claim 56, wherein the carboxypeptidase is CPE.

59. The method of claim 54, wherein the host cells comprises a mutated CPE with at least one mutation at a position selected from a group consisting of 72H, 75E, 147R, 192H, 202S, 243Y, and 296E of the sequence of SEQ ID NO: 9.

60. The method of claim 55, wherein the mutated CPE is catalytically-inactive, and wherein the catalytically-inactive CPE protein binds to but does not hydrolyze the protein.

61. The method of claim 54, wherein the protein is produced continuously.

62. The method of claim 58, wherein expression and/or activity of the endogenous CPE protein is reduced or eliminated.

63. A modified mammalian host cell, wherein the modified mammalian host cell genome comprises a mutated CPE gene encoding a mutated CPE.

64. The modified mammalian host cell of claim 62, wherein expression and/or activity of the CPE is reduced or eliminated.

65. The mammalian host cell of claim 61, wherein the mutated CPE is catalytically-inactive.

66. The mammalian host cell of claim 61, wherein the mutated CPE binds to but does not hydrolyze protein.

67. A polynucleotide encoding a mutated CPE, wherein the mutated CPE is catalytically-inactive.

68. The polynucleotide of claim 67, wherein the mutated CPE binds to but does not hydrolyze a protein.

69. The polynucleotide of claim 67 or 68, wherein the mutated CPE comprises at least one mutation at a position selected from a group consisting of 72H, 75E, 147R, 192H, 202S, 243Y, and 296E of the sequence of SEQ ID NO: 9

70. A mutated CPE derived from the sequence of SEQ ID NO:9, wherein the mutated CPE is catalytically-inactive.

71. The mutated CPE of claim 70, wherein the mutated CPE binds to but does not hydrolyze a protein.

72. The mutated CPE of claim 70 or 71, wherein the mutated CPE comprises at least one mutation at a position selected from a group consisting of 72H, 75E, 147R, 192H, 202S, 243Y, and 296E of the sequence of SEQ ID NO: 9.


SEQUENCE LISTING (32 sequences)

SEQ ID NO: 1 - protein, 110 aa, Homo sapiens
MALWMRLLPLLALLALWGPDPAAAFVNQHLCGSHLVEALYLVCGERGFFYTKPTRREAEDLQVGQVELGGGPGAGSLQPLALEGSLQKRGIVEQCCTSICSLYQLENYCN

SEQ ID NO: 2 - protein, 305 aa, Homo sapiens
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSRRKRFVNQHLCGSHLVEALYLVCGERGFFYTKPTRREAEDLQVGQVELGGGPGAGSLQPLALEGSLQKRGIVEQCCTSICSLYQLENYCN

SEQ ID NO: 3 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCG

SEQ ID NO: 4 - protein, 305 aa, Homo sapiens
METDTLLLWVLLLWVPGSTGDYKDEHHHHHHGGSQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDGGGSRRKRFVNQHLCGSHLVEALYLVCGERGFFYTPKTRREAEDLQVGQVELGGGPGAGSLQPLALEGSLQKRGIVEQCCTSICSLYQLENYCG

SEQ ID NO: 5 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 6 - protein, 638 aa, Homo sapiens
MKGGCVSQWKAAAGFLFCVMVFASAERPVFTNHFLVELHKGGEDKARQVAAEHGFGVRKLPFAEGLYHFYHNGLAKAKRRRSLHHKQQLERDPRVKMALQQEGFDRKKRGYRDINEIDINMNDPLFTKQWYLINTGQADGTPGLDLNVAEAWELGYTGKGVTIGIMDDGIDYLHPDLASNYNAEASYDFSSNDPYPYPRYTDDWFNSHGTRCAGEVSAAANNNICGVGVAYNSKVAGIRMLDQPFMTDIIEASSISHMPQLIDIYSASWGPTDNGKTVDGPRELTLQAMADGVNKGRGGKGSIYVWASGDGGSYDDCNCDGYASSMWTISINSAINDGRTALYDESCSSTLASTFSNGRKRNPEAGVATTDLYGNCTLRHSGTSAAAPEAAGVFALALEANLGLTWRDMQHLTVLTSKRNQLHDEVHQWRRNGVGLEFNHLFGYGVLDAGAMVKMAKDWKTVPERFHCVGGSVQDPEKIPSTGKLVLTLTTDACEGKENFVRYLEHVQAVITVNATRRGDLNINMTSPMGTKSILLSRRPRDDDSKVGFDKWPFMTTHTWGEDARGTWTLELGFVGSAPQKGVLKEWTLMLHGTQSAPYIDQVVRDYQSKLAMSKKEELEEELDEAVERSLKSILNKN

SEQ ID NO: 7 - protein, 476 aa, Homo sapiens
MAGRGGSALLALCGALAACGWLLGAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 8 - protein, 476 aa, Homo sapiens
MAGRGGSALLALCGALAACGWLLGAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVALSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 9 - protein, 434 aa, Homo sapiens
LQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 10 - protein, 21 aa, Homo sapiens
GIVEQCCTSICSLYQLENYCN

SEQ ID NO: 11 - protein, 30 aa, Homo sapiens
FVNQHLCGSHLVEALYLVCGERGFFYTKPT

SEQ ID NO: 12 - protein, 21 aa, Homo sapiens
GIVEQCCTSICSLYQLENYCN

SEQ ID NO: 13 - protein, 30 aa, Homo sapiens
FVNQHLCGSHLVEALYLVCGERGFFYTDKT

SEQ ID NO: 14 - protein, 21 aa, Homo sapiens
GIVEQCCTSICSLYQLENYCG

SEQ ID NO: 15 - protein, 32 aa, Homo sapiens
FVNQHLCGSHLVEALYLVCGERGFFYTPKTRR

SEQ ID NO: 16 - protein, 25 aa, Homo sapiens
MGVLLTQRTLLSLVLALLFPSMASM

SEQ ID NO: 17 - protein, 16 aa, Homo sapiens
MKCLLYLAFLFIGVNC

SEQ ID NO: 18 - protein, 21 aa, Homo sapiens
METDTLLLWVLLLWVPGSTGD

SEQ ID NO: 19 - protein, 19 aa, Homo sapiens
MGWSCIILFLVATATGVHS

SEQ ID NO: 20 - protein, 17 aa, Homo sapiens
MRAWIFFLLCLAGRALA

SEQ ID NO: 21 - protein, 21 aa, Homo sapiens
MWWRLWWLLLLLLLLWPMVWA

SEQ ID NO: 22 - protein, 22 aa, Homo sapiens
MDMRVPAQLLGLLLLWLRGARC

SEQ ID NO: 23 - protein, 16 aa, Homo sapiens
MPLLLLLPLLWAGALA

SEQ ID NO: 24 - protein, 23 aa, Homo sapiens
MDAMKRGLCCVLLLCGAVFVSPS

SEQ ID NO: 25 - protein, 18 aa, Homo sapiens
MAFLWLLSCWALLGTTFG

SEQ ID NO: 26 - protein, 15 aa, Homo sapiens
MNLLLILTFVAAAVA

SEQ ID NO: 27 - protein, 20 aa, Homo sapiens
MYRMQLLSCIALSLALVTNS

SEQ ID NO: 28 - protein, 17 aa, Homo sapiens
MGVKVLFALICIAVAEA

SEQ ID NO: 29 - protein, 16 aa, Homo sapiens
MKWVTFISLLFSSAYS

SEQ ID NO: 30 - protein, 16 aa, Homo sapiens
MKTIIALSYIFCLVLG

SEQ ID NO: 31 - protein, 24 aa, Homo sapiens
MALWMRLLPLLALLALWGPDPAAA

SEQ ID NO: 32 - protein, 16 aa, Homo sapiens
MKPIFLVLLVVTSAYA